(19) United States
(12) Patent Application Publication
(10) Pub. No.: US 2010/0221347 A1
(43) Pub. Date: Sep. 2, 2010

(54) ENHANCING SOLUTE TRANSPORT WITHIN A TISSUE SCAFFOLD

(76) Inventors: Erik L. Ritman, Rochester, MN (US); Jorn Op Den Buus, Rochester, MN (US); Kai-Nan An, Rochester, MN (US); Armando Manduca, Rochester, MN (US); Dan Dragomir Daescu, Rochester, MN (US); Virginia M. Miller, Rochester, MN (US); Zeljko Bajzer, Rochester, MN (US); Mair Zamir, Ontario (CA)

Correspondence Address: FISH & RICHARDSON P.C., PO BOX 1022, MINNEAPOLIS, MN, 55440-1022, US

(21) Appl. No.: 12/707,595

(22) Filed: Feb. 17, 2010

Related U.S. Application Data
(60) Provisional application No. 61/153,567, filed on Feb. 18, 2009.

(51) Int. Cl. A61K 9/14; A61K 35/34; A61P 9/00; A61P 19/00; A61P 19/02; A61P 25/00; A61P 3/10

(52) U.S. Cl. 424/489; 424/93.7

(57) ABSTRACT

This document provides materials and methods related to tissue scaffolds for use in replacing or augmenting various tissues in the body. For example, flexible tissue scaffolds with controlled pore geometry and methods of enhancing solute transport using rhythmic compression (e.g., 1.0 Hz) of tissue scaffolds are provided

### CROSS-REFERENCE TO RELATED APPLICATION

This application claims priority to U.S. Provisional Application No. 61/153,567, filed Feb. 18, 2009, the contents of which are incorporated by reference in their entirety herein.

### STATEMENT AS TO FEDERALLY FUNDED RESEARCH

This invention was made with government support under EB000305 awarded by the National Institutes of Health. The government has certain rights in the invention.

### BACKGROUND

1. Technical Field

This document provides materials and methods related to tissue scaffolds for use in replacing or augmenting various tissues in the body. For example, flexible tissue scaffolds with controlled pore geometry and methods of enhancing solute transport using rhythmic compression (e.g., 1.0 Hz) of tissue scaffolds are provided.

2. Background Information

Prostheses are devices that are used to support or replace a body part lost by trauma, disease, or defect. Improved prostheses are required to meet the needs of the aging population.

Recently, there has been a shift from replacing lost body parts to regenerating damaged organs or tissues. Tissue engineering techniques have the potential to create tissues and organs de novo, using cells integrated into a three-dimensional scaffold.

### SUMMARY

This document provides materials and methods related to tissue scaffolds for use in replacing or augmenting various tissues in the body. For example, flexible tissue scaffolds with varying pore geometry and methods of rhythmically compressing tissue scaffolds to increase solute transport within the scaffold are provided. In tissue engineering, scaffolds can provide a three-dimensional template for cells. The cells can attach to and grow onto the surface of the pores in a tissue scaffold. The pores can simultaneously allow for supply of nutrients and oxygen to the attached cells, and removal of metabolic waste from the cells. In some cases, the materials and methods described herein can be used to increase solute convection deep into a tissue scaffold and provide an enhanced environment for cell survival at increased depths (i.e. >>1 mm) before ingrowth of microvessels into a tissue scaffold. For example, tissue scaffolds configured to work to increase solute transport in response to an applied force (e.g., a compressive or expansive force). In some cases, a tissue scaffold can be configured to provide a net unidirectional fluid flow, which can be modulated by compression frequency. The methods and materials described herein can be used in designing tissue scaffolds with appropriate porosity, pore connectivity, elasticity and compliance, flow characteristics, strength and durability as required by the application of a tissue scaffold.

In general, one aspect of this document features a method for supporting tissue growth within a mammal. The method can comprise, or consist essentially of, implanting a tissue scaffold into a location in the mammal. The location can provide a compressive or expansive force to the tissue scaffold. The force can be generated from a natural body movement or body function. The mammal can be a human. The tissue scaffold can include a population of cells. The cells can be selected from among stem cells, preadipocytes, glia, fibroblasts, myocytes, and osteocytes. The location can be selected from among the heart, intestines, vasculature, knee, hip, or jaw. The force can be applied cyclically. The frequency of the force can be equal to or greater than about 1.0 Hz. The force can enhance solute transport within the tissue scaffold. The body function can comprise, or consist essentially of, beating of the mammal's heart, pulsation of the mammal's arteries, or peristaltic motion of the mammal's intestines. The body movement can comprise, or consist essentially of, exercise, walking, running, or chewing.

In another aspect, this document features a method for supporting tissue growth within a mammal. The method can comprise, or consist essentially of, implanting a tissue scaffold that is responsive to external stimulation into a mammal. The external stimulation can provide a compressive or expansive force to the tissue scaffold. The tissue scaffold can be responsive to electrical current stimulation. The tissue scaffold can include magnetic particles and be responsive to magnetic field stimulation. The tissue scaffold can include a population of cells. The cells can be selected from among stem cells, islet cells, preadipocytes, glia, fibroblasts, myocytes, and osteocytes. The method can include stimulating the tissue scaffold. The frequency of the stimulation can be equal to or greater than about 1.0 Hz. The stimulation can enhance solute transport within the tissue scaffold. The mammal can be a human.

In another aspect, this document features a method for supporting tissue growth within a mammal. The method can comprise, or consist essentially of, implanting a tissue scaffold into a location in the mammal that is accessible to externally applied massage. The massage can provide a compressive or expansive force to the tissue scaffold. The mammal can be a human. The tissue scaffold can include a population of cells. The cells can be selected from among stem cells, preadipocytes, glia, fibroblasts, myocytes, and osteocytes. The location can be selected from among the limbs, skin, gums, and jaw. The massage can be performed by a mechanical massage device. The frequency of the force provided by the massage can be equal to or greater than about 1.0 Hz. The massage can enhance solute transport within the tissue scaffold. The method can include massaging the location.

In another aspect, this document features a method for supporting tissue growth within a mammal. The method comprises, or consists essentially of, implanting a tissue scaffold into a location in the mammal, wherein the location provides a compressive or expansive force to the tissue scaffold, wherein the force is generated from a natural body movement or body function, and wherein the tissue scaffold comprises concentric layers. The mammal can be a human. The tissue scaffold can comprise a population of cells. The tissue scaffold can comprise microspheres. The microspheres can be selected from the group consisting of solid microspheres, porous microspheres, and degradable microspheres. The tissue scaffold can comprise a porous geometry for solute transport. The location can be selected from the group consisting of the heart, intestines, vasculature, knee, hip, and jaw. The force can be applied cyclically. The frequency of the force can be equal to or greater than about 1.0 Hz. The force can enhance solute transport within the tissue scaffold. The body function can comprise beating of the mammal's heart or pulsation of the mammal's arteries.

In another aspect, this document features a method for supporting tissue growth within a mammal. The method comprises, or consists essentially of, injecting an injectable tissue scaffold material into a location in the mammal, wherein the location is substantially free from a compressive or expansive force, wherein the injectable tissue scaffold material forms a porous geometry for solute transport. The injectable tissue scaffold can comprise microspheres. The microspheres can be selected from the group consisting of solid microspheres, porous microspheres, and degradable microspheres. The location can be within a vertebral body.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic of the scaffold fabrication technique.

FIG. 2 is schematic of the experimental set-up.

FIG. 3 shows projection X-ray images of uncompressed scaffold (left) and the scaffold at maximal level of compression (right).

FIG. 4 shows consecutive projection X-ray images taken during passive removal (upper panel) and upon deformation (lower panel).

FIG. 5: Panel A is a plot of the mean X-ray attenuation along the channel axis during 13% compression v. position (microns) at different time points. Panel B is a plot of relative average of NaI concentration in the scaffold channel during passive removal and upon deformation v. time (seconds).

FIG. 6 is a plot of the exponential function with two rate constants fitted to the experimental data obtained during the compression-induced removal of NaI, showing the presence of a fast and a slow component.

FIG. 7 shows micro-CT images of contrast agent distribution in a deformable cubic scaffold with a single channel after fifty cycles of compression at 1.0 Hz.

FIG. 8 is a plot of relative NaI concentration v. time showing the compression-induced removal curve is slightly right-shifted if the time required to record the X-ray images is taken into account.

FIG. 9 is a schematic showing nutrient transfer under static conditions and convective transport induced by repetitive mechanical deformation of the scaffold.

FIG. 10 is a schematic of the solid freeform fabrication method of manufacturing scaffolds with programmable pore shapes.

FIG. 11 is a photograph of the experimental set-up.

FIG. 12 is a schematic of boundary conditions for modeling the fluid structure relationship in a mechanically deformed scaffold.

FIG. 13 shows X-ray micro images of 4.5×4.5×4.5 mm³ cubic tissue scaffolds with single flow channels in the shape of a circular cylinder, a spheroid, and an elliptic cylinder with its major axis perpendicular, and parallel to the strain direction. The scaffolds are shown in the uncompressed state, the maximally compressed state (˜10% of the original scaffold height), after injection of X-ray absorbing tracer (at t=0), and after 300 compressions at 1.0 Hz (at t=300 seconds).

FIG. 14 is plot of the average contrast agent concentration inside the channel as a function of time relative to the concentration at t=0, for each channel shape. Experimental X-ray measurements (dots with error bars) are compared to numerical Fluid-Structure Interaction (FSI) modeling results.

FIG. 15 shows representative micro-CT images (20 μm voxel size) of the specimens with pores in the form of a circular cylinder (1.5 mm), spheroid and elliptic cylinder. White is scaffold material; black is air.

FIG. 16 shows projection X-ray images of the uncompressed specimen and the specimen at maximal level of compression. Side views are shown for the 1.5 mm diameter circular cylinder (top left) and spheroid channel (bottom left). The elliptical channel is shown from the front with the compression perpendicular to (top right) and in parallel with (bottom right) the major axis of the elliptical cross-section.

FIG. 17 shows consecutive projection X-ray images taken during compression-induced and passive removal of NaI from the specimen channels. Compression was performed at 1.0 Hz, such that 300 seconds correspond to 300 compressions. A. Circular pore (1.5 mm diameter). B. Spheroid pore. C. Elliptic pore.

FIG. 18 is a plot of relative iodine concentration in the specimen channel upon passive removal for the spheroid channel and upon deformation-induced removal for the spheroid channel, 1.50 mm circular cylindrical channel and elliptical channel. Data represent means±SD for n=5.

FIG. 19 is a bar graph showing the fraction of remaining iodine concentration after 300 seconds of passive transport (white bars) or deformation-induced transport (black bars). Data represent means±SD for n=5. *: significantly different compared to passive removal (P<0.05). #: significantly different compared to 0.5 mm diameter channel (P<0.05).

FIG. 20 is a plot of the correlation between channel compression and remaining fraction of iodine after 300 s of compressions for the different channel shapes. Linear regression yielded a significant relationship (y=−0.0131 x+0.9735, R²=0.9803). Note that the 2.0 mm circular channel was excluded from the regression.

FIG. 21 shows Grayscale images of the measured intensities right after injection, subtracted from the measured intensity after 300 compression cycles. White pixels mean that contrast agent was completely removed at this position. Difference images are shown for the 1.5 mm circular cylinder pore (top left), the spheroid (top right), and the elliptic pore in both directions (bottom).

FIG. 22 shows projection X-ray images of an imaging phantom with simple pore network with interconnected channels in one plane in the uncompressed state and at maximal level of compression (16.6% of the original height) (upper panel). Consecutive projection X-ray images taken during compression-induced and passive removal of NaI from the phantom channels (lower panel).

FIG. 23 is a schematic illustrating that scaffold pore geometry can be modified to control the solute transport rate induced by cyclic loading of the scaffold.

FIG. 24 is shows tracer concentration distributions in a 5×5×5 mm³ scaffold containing circular cylindrical flow channels that are interconnected in a plane. The scaffold was compressed with an amplitude of 15% at 1.0 Hz.

FIG. 25 is a plot of the average concentration of tracer in the scaffold with interconnected channels (diffusion only, 10%, and 15% compression at 1.0 Hz).

FIG. 26 shows a computational fluid dynamics simulation (CFD) mesh of an asymmetric pore system comprised of a pumping chamber connected to nozzle and diffuser elements with openings to the nutrient-rich surrounding environment (a reservoir in a bioreactor or the interstitial fluid after implantation). The scaffold (not shown) surrounds the pumping chamber and the nozzle/diffuser elements. Upon cyclic pumping of such a scaffold, a net fluid flow through the scaffold may be established.

FIG. 27 is a plot of the magnitude of the net fluid flow v. pump frequency, as demonstrated by means of CFD simulations. The net fluid flow is in the direction of the diverging channels.

FIG. 28 is a schematic diagram of the model. (A) A deformable scaffold is immersed in a fluid reservoir underneath a compression device. The pore of the scaffold is initially filled with contrast agent. Cycles of (B) compression and (C) release are applied to the scaffold, inducing a convective fluid flow in the scaffold pore, thereby transporting the tracer from the pore into the surrounding fluid reservoir.

FIG. 29 shows the modeled scaffold pore geometries. (A) Circular cylinder with channel diameter d1. (B) Elliptic cylinder with minor axis d1 and major axis d2. (C) Spheroid pore with opening diameter d1 and maximum diameter d2. See Table 3 for dimensions.

FIG. 30 represents discretization of the solid and fluid domains for numerical solution of the model partial differential equations using the finite element and finite volume methods. (A) Solid mesh and (B) fluid mesh (pore+reservoir) of a scaffold with 1.38 mm circular cylindrical pore. (C) Solid mesh and (D) fluid mesh of a scaffold with spheroid pore.

FIG. 31 is a schematic diagram of the boundary conditions used in the model.

FIG. 32 shows X-ray data and CFD model result compared during passive (gravitation induced) removal of contrast agent from the scaffold channel. (A) Distribution of contrast agent inside the channel. (B) Average concentration of iodine in the channel.

FIG. 33 shows X-ray data and CFD model result compared during compression-induced removal of contrast agent from the scaffold channel. (A) Circular cylinder. (B) Elliptic cylinder with minor axis in the strain direction. (C) Spheroid.

FIG. 34 shows a model (A) predicted relative iodine concentration in the channel compared to the X-ray data. The error bars result from n=5 repeated experiments. (B) Percentage of iodine removed as predicted by the computational model compared to X-ray data for the different channel shapes at t=100 s. Notice the excellent agreement between model and data.

FIG. 35 shows (A) X-ray projection images of diffusion of sodium iodide in glycerol at different time points. The glycerol is dark and the iodine is light. (B) Fit of the analytical diffusion equation to calculate the diffusion coefficient.

FIG. 36 is a model depicting (left panel) a cross-section of the conduit. The white sector in the wall is magnified in the right panel. (right panel) Schematic of a sector of the conduit wall. The microspheres are fixed and the gaps between them form the spaces that can be invaded by blood vessels and cells. The inner spheres are 20 μm diameter and the outer are 140 μm diameter. The outer spheres are stiffer than the inner.

FIG. 37 is a schematic of use of injectable scaffold components to micro-“inflate” a collapsed vertebra. The double syringe technique allows delivery of two types of microspheres (100-200 μm diameter, shown as clear and black dots) which fuse on contact by virtue of ‘click’ chemistry. This will form a porous scaffold which has a labyrinth of interconnected pores with a limited range of pore diameters. Variations in adhesion rates and “mixing” of the two types of spheres and “packing” will be the main focus of our investigations.

FIG. 38 is a model predicted distribution of solute at t=100 s inside a scaffold with interconnected circular channels (d=1.0 mm) upon compression-induced cyclic deformation with 15% strain at 1.0 Hz.

### DETAILED DESCRIPTION

This document provides materials and methods related to tissue scaffolds for use in replacing or augmenting various tissues in the body. For example, flexible tissue scaffolds with controlled pore geometry and methods of enhancing solute transport using rhythmic compression (e.g., 1.0 Hz) of tissue scaffolds are provided.

A tissue scaffold can be an artificial structure capable of supporting three-dimensional tissue formation. Any appropriate mechanically deformable material can be used as a tissue scaffold (e.g., a variety of natural, synthetic, and biosynthetic polymers). In some cases, a tissue scaffold can comprise a biodegradable, crosslinkable, and/or biocompatible polymer (e.g., Poly(esters) based on polylactide (PLA), polyglycolide (PGA), polycaprolactone (PCL), and their copolymers).

Any appropriate cross-linking agent can be used to form chemical links between molecule chains to form a three-dimensional tissue scaffold. For example, crosslinked PPF and PCLF have distinct characteristics resulting from different densities of crosslinkable moieties on the polymer backbones. Crosslinked PPF can have an average tensile modulus E=1.3 GPa while crosslinked PCLF can have an average tensile modulus E=2.1 MPa. Material properties, particularly mechanical properties, can be modulated through varying the composition of polymer components of the scaffold materials. PPF/PCLF blends with PPF weight composition of about 25% and PCLF composition of about 75% can be used to manufacture deformable scaffolds as described herein.

Deformable scaffolds with programmed flow channel geometries can be fabricated using a solid freeform fabrication (SFF) technique, such as a combination of three-dimensional printing and injection molding as described elsewhere (Lee et al., Tissue Eng, 12: 2801-2811 (2006)). For example, SFF can include designing an injection mold using computer-aided design (CAD) software, three-dimensional printing of the mold, injecting and cross-linking of an elastic (biodegradable/biocompatible) polymer into the mold, and removing the mold material by mechanical, thermal, or chemical treatment without affecting the polymer of interest. The CAD files can be used to generate temporary negative molds, which are injected with a biodegradable polymer to cast a tissue scaffold. In some cases, SFF methods can be used to create synthetic scaffolds featuring interconnected pores and programmed pore geometries. Tissue scaffolds featuring complex pore structures e.g., interconnected channels, tortuous (non-straight) channels, and pores with different shapes can be manufactured using the methods and materials provided herein. In some cases, such complex pore structures can be obtained using solid spheres, porous microspheres, and/or degradable microspheres.

SFF methods can be used to maintain the mechanical strength of a tissue scaffold (e.g., by controlling porosity), and permit mechanical properties (e.g., stiffness, yield limit, etc.) to be tailored for specific applications. For example, SFF methods can be used to fabricate a scaffold capable of acting as a load bearing structure during tissue regrowth (e.g., knee cartilage, vascular, or cardiac tissue). In some cases, scaffold fabrication methods can be automated.

During fabrication, a porous tissue scaffold can be embedded with magnetic particles (e.g., ferromagnetic or paramagnetic particles). For example, scaffolds embedded with magnetic particles can form actuable structures. In some cases, such scaffolds can be deformed remotely by application of a magnetic field. See, e.g., Cox et al., U.S. Pat. Pub. No. 2007/0151202 and Mack et al., J. Mater. Sci., 42: 6139-6147 (2007).

Acellular tissue scaffolds or cell-populated tissue scaffolds can be used with the methods described herein. In some cases, a tissue scaffold can be seeded with a population of cells before implantation. Any appropriate cell type, such as naïve or undifferentiated cell types, can be used to seed a tissue scaffold. For example, a population of cells (e.g., stem cells, cardiomyocytes, myocytes, osteocytes, fibroblasts, glia, or preadipocytes) can be cultured on a tissue scaffold. In some cases, autologous stem cells from any tissue source (e.g., skin, bone, synovium, fat, marrow, or muscle) can be used. Any appropriate method for isolating and collected cells for seeding can be used.

Upon fabrication of a tissue scaffold, the polymer material can be embedded with bioactive molecules, e.g., to be transported into surrounding tissue, or distributed to cells inside a scaffold. Bioactive agents can promote wound healing and/or angiogenesis in and around an implanted tissue scaffold, for example. Appropriate bioactive agents can include polypeptides (e.g., growth factors ((VEGF), transforming growth factor-β (TFG-β), and fibroblast growth factor (FGF)), cytokines, and antibodies), antimicrobial agents (e.g., antibiotics and antifungal agents), analgesic/anti-inflammatory agents (e.g., NSAIDs and steroidal agents), immunomodulators (e.g., cyclosporine and interferon), and/or local anesthetics (e.g., lidocaine and procaine), which can be embedded in the scaffold. See, e.g., Rocha et al., Biomaterials 29: 2884-2890 (2008). In some cases, polypeptides or other signal molecules can be released by cells (e.g., engineered cells) embedded in the scaffold (e.g., gene therapy). See, e.g., El-Ayoubi et al., Tissue Eng, Part A (2008). Transport of embedded agents can be enhanced by cyclical distortion of a tissue scaffold. In some cases, the geometry of tissue scaffold pores can be tailored to yield a specific transport pattern of an embedded agent. Repetitive deformation of a tissue scaffold can augment solute transport as compared to a static tissue scaffold (i.e., a scaffold under conditions of diffusional transport). See e.g., Op Den Buijs et al., “High resolution X-ray imaging of dynamic solute transport in cyclically deformed porous tissue scaffolds,” SPIE Medical Imaging: Physiology, Function and Structure from Medical Images, San Diego, Calif., 2008. For example, a tissue scaffold with one or more pores can be subjected to a cyclically varying load (e.g., rhythmic compressions or other deformations). In some cases, a cyclic load can be the result of intrinsic rhythmic deformation of the surrounding tissue (e.g., beating of the heart, pulsation of arteries, peristaltic motion of the intestines, periodic knee cartilage loading during walking, and muscle contraction/relaxation cycles), or repetitive mechanical forces applied external to a part of the body in close proximity to an implantation site. The mechanical distortion of the tissue surrounding a tissue scaffold can be induced by forms of rehabilitation therapy (e.g., massage therapy, exercise, and/or electrical muscle stimulation). See, e.g., Goats, Br J Sports Med 28: 153-156 (1994) and U.S. Pat. App. Pub. No. 2007/0270917. Cyclic loading can cause a corresponding rhythmic distortion of the pores of a tissue scaffold, resulting in a cyclic motion of fluid present within the scaffold pores.

In some cases, the cyclic fluid flow can enhance the dynamic mixing of components in the surrounding interstitial fluid or blood to bring nutrients closer to the scaffold inlets, and pump waste products outside a tissue scaffold. For example, during the part of the loading cycle in which the pore volume effectively decreases (i.e., during compression or stretching), fluid containing waste products of cells and possibly toxic degradation products of the scaffold material present in the scaffold pores can be squeezed out of the scaffold pores and mixed with the interstitial fluid (or blood) surrounding a tissue scaffold. Upon subsequent recoil of the scaffold to its original shape, ‘fresh’ fluid with no (or a low concentration of) waste products can flow back into the scaffold pore, thereby decreasing the concentration of waste and degradation products in the fluid inside the scaffold pores. During the part of the loading cycle in which the pore volume effectively increases (i.e., during recoil after compression or stretching), interstitial fluid rich in nutrients and oxygen can be transported from the surrounding tissue (or blood) into the scaffold. This fluid can mix with the nutrient- and oxygen-deficient fluid inside the scaffold pores, thereby increasing the concentration of nutrients and oxygen in the fluid inside the scaffold pores.

Solute convection due to rhythmic pore deformation and the resulting cyclic fluid motion can be related to the geometry of the pores. For example, different pore geometries can lead to different effective pore volume changes during the cyclic loading, even when the same amount of deformation is applied. The cross-sectional shape of the pores (e.g., a circular cross-section vs. an elliptical cross-section), the diameter of the pore cross-sectional area, the orientation of cross-sectional asymmetries with respect to the direction of the cyclic strain, and any interconnections between pores, can influence convective solute transport. The pore geometry can be tailored to yield the specific nutrient transport rates and depths, as required by the application of a tissue scaffold.

In symmetric flow channels, such as cylinders with circular or elliptic cross-sections that have constant diameters along the channel, cyclic pumping of a scaffold can induce a bi-directional motion of fluid in the flow channels, thereby enhancing the spreading and mixing of nutrients and waste products as described above. In some cases, a combination of cone-shaped channels and cylindrical or spherical pumping chambers can be created to alter the direction of flow. For example, upon cyclic compression (>1.0 Hz) fluid can be preferentially pumped in one direction, thereby resulting in a frequency-dependent net fluid flow across the scaffold. The unidirectional fluid flow can transport solutes deeper into a tissue scaffolds and/or obtain spatially uniform distribution of solutes. In some cases, unidirectional flow can oppose physiological conditions restricting flow in a tissue scaffold, to pump the nutrients from the source deep into a tissue scaffold (e.g., more than 1.0 mm from the surface of the tissue scaffold).

Any mammal can have a tissue scaffold implanted for supporting tissue growth using the materials and methods provided herein. For example, a human, mouse, cat, dog, or horse can have a tissue scaffold that supports tissue growth implanted for regeneration or support of a damaged tissue. Any appropriate tissue can be replaced using the methods and materials described herein. Mechanically active tissues that are appropriate for tissue-engineering using deformable scaffolds include blood vessels, cardiac muscle and heart valves, bone and cartilage, tendons and ligaments, nerves, adipose tissue (e.g., for breast augmentation or restoration after mastectomy), and periodontal structures. For example, many tissues (e.g., knee cartilage, tendons, cardiac and vascular tissues) undergo forms of cyclic loading with strains up to about 30% (e.g., about 5-10%, 10-20%, and 20-30%). See, e.g., (Teske et al., Cardiovasc. Ultrasound, 5: 27 (2007); Bingham et al., Rheumatology 47(11):1622-1627 (2008); Liang et al., J. Biomech., 41(14):2906-2911 (2008); and, Stafilidis et al., Eur. J. Appl. Physiol., 94: 317-322 (2005). Strains of these magnitudes can induce significant solute convection in scaffolds used to replace or support such tissues with appropriate pore geometry. In some cases, deformable scaffolds can be appropriate for insulin delivery systems in diabetic patients.

In some cases, a tissue scaffold can be injected into a mammal. For example, a tissue scaffold can be injected in liquid form into an intended location of a mammal. Any appropriate bodily organ or tissue of a mammal can be injected according to the methods and materials described herein. For example, bone can be injected with an injectable tissue scaffold. In some cases, an injectable tissue scaffold can produce scaffolds that have interconnected pores suitable for maintaining viable cells. In some cases, an injectable tissue scaffold can be ultimately replaced by ingrowing tissue.

Any appropriate technique can be used to measure mechanical deformation of a tissue scaffold in vitro or an implanted tissue scaffold in vivo. Mechanical strain induced by compression can be measured in vivo using imaging systems such as ultrasound and magnetic resonance. Three dimensional micro-CT or cryostatic micro-CT as described elsewhere (Kantor et al., Scanning 24: 186-190 (2002)) can be used to image compression-induced deformation of a tissue scaffold in vitro.

Any appropriate technique can be used to quantify fluid flow in a tissue scaffold. The term “enhanced” as used herein with respect to solute transport is any transport that is increased relative to passive transport of a solute (e.g., diffusion) in a biological fluid (e.g., plasma). Enhanced solute transport can have an increased rate and/or increased depth of solute transport (e.g., to scaffold depths>0.1 mm). In some cases, an enhanced level of solute transport can be any detectable level of solute transport.

Measurement of solute transport in vivo can be simulated in an experimental setting in vitro. For example, the rate and depth of solute transport can be measured using a contrast agent to simulate a solute in a biological fluid. In addition to the methods described above, an X-ray system with a spectroscopic X-ray source and a detector can provide information about fluid dynamics in a tissue scaffold in vitro. In some cases, physiologically equivalent compressions (e.g., 1.0 Hz) can be simulated using a compression device to induce deformations in a scaffold in a fluid reservoir and X-ray images collected over time can be analyzed to provide rate and depth of solute transport in a tissue scaffold with and without cyclic compressions.

Solute transport can also be determined by assessing ingrowth of cells and microvessels into deep layers of a tissue scaffold. For example, a tissue scaffolds can be seeded with cells in vitro and imaged (e.g., using fluorescent microscopy) at specific intervals during cell culture (e.g., at T=0, 5, 10, and 20 days). In some cases, a tissue scaffold as described herein can support a population of cells at a greater depth from a scaffold surface and/or for a longer period of time in culture than the same cell-type seeded on a corresponding tissue scaffold that is not subjected to cyclic compressions (e.g., deeper than about 200 μm and/or more than 21 days).

The invention will be further described in the following examples, which do not limit the scope of the invention described in the claims.

### EXAMPLES

### Example 1

### High-Resolution Imaging of Dynamic Solute Transport in Cyclically Deforming Porous Scaffolds

An X-ray based imaging method to quantify solute transport induced by mechanical compression at 20 μm pixel resolution was developed and applied to flexible biodegradable scaffolds with a controlled pore structure. Using a contrast agent as a surrogate for non-radiopaque nutritional solutes (Jorgensen et al., Am. J. Physiol. Heart Circ. Physiol., 275: H1103-H1114 (1998)), this technique was used to image opaque specimens at micrometer spatial resolution, and collect quantitative information about the local concentration of an X-ray absorbing contrast agent.

### Material and Methods

Scaffold Material

Polypropylene fumarate (PPF) and polycaprolactone fumarate (PCLF) are biodegradable, crosslinkable, and biocompatible. Peter et al., J. Biomed. Mater. Res., 41: 1-7 (1998); Yaszemski et al., Biomaterials, 17: 2127-2130 (1996); and, Jabbari et al., Biomacromolecules, 6: 2503-2511 (2005). Crosslinked PPF and PCLF have distinct characteristics because of different density of crosslinkable segment on the polymer backbone. Crosslinked PPF is a stiff material with an average tensile modulus E of 1.3 GPa while crosslinked PCLF is a flexible material with E=2.1 MPa. PPF is a promising candidate injectable biomaterial to substitute autologous or allograft bone, especially for load-bearing purposes. PCLF can be used to fabricate single-lumen and multi-channel tubes for guiding axon growth in peripheral nerve repair. Material properties, particularly mechanical properties, can be efficiently modulated through varying the composition of PPF in PPF/PCLF blends. See, e.g., Wang et al., Biomacromolecules, 9(4): 1229-1241 (2008).

PPF with a number-average molecular weight (Mn) of 3460 g/mol and a weight-average molecular weight (Mw) of 7910 g/mol and PCLF with an Mn of 3520 g/mol and an Mw of 6050 g/mol were used to prepare PPF/PCLF blends. One PPF/PCLF blend with PPF weight composition of 25% and PCLF composition of 75% was prepared by first dissolving PPF and PCLF sufficiently in a co-solvent methylene chloride (CH₂Cl₂) and then evaporating the solvent in a vacuum oven. PPF and PCLF were polymerized in our laboratory (as described in Wang et al., Biomacromolecules, 7: 1976-1982 (2006). The PCLF sample was synthesized using α,ω-telechelic PCL diol with a nominal Mn of 530 g/mol and fumaryl chloride in the presence of potassium carbonate (as described in Wang et al., Biomaterials, 27: 832-841 (2006)).

Scaffold Fabrication

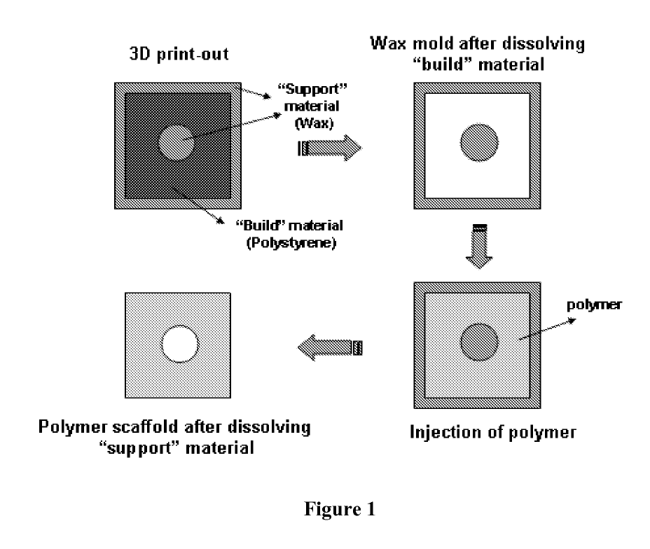

Biodegradable scaffolds with a programmable pore structure were fabricated (as described elsewhere (Lee et al., Tissue Eng, 12: 2801-2811 (2006)). Computer-aided design (CAD) models were created using Solidworks (SolidWorks Corp., Concord, Mass.), meshed into stereolithography (STL) files, and converted to 2D sliced data files with a thickness of 76 μm using the ModelWorks software (Solidscape Corp., Merrimack, N.H.). The 3D phase-change ink jet printer, PatternMaster, was used to create 3D scaffolds by printing PTM files layer-by-layer with a build material (polystyrene) and a support material (wax). After printing, the polystyrene was dissolved by immersing the printed scaffolds into acetone for 30 minutes to obtain wax molds (FIG. 1). Subsequently, the wax molds were put into a Teflon holder and PPF/PCLF polymerizing mixture was injected under 100 mmHg vacuum. The PPF/PCLF polymer blend was then crosslinked by free radical polymerization with benzoyl peroxide (BPO), dimethyl toluidine (DMT), 1-vinyl-2-pyrrolidinone (NVP), and methylene chloride as free radical initiator, accelerator, crosslinker, and diluent, respectively. 100 μL of initiator solution (50 mg of BPO in 250 μL of NVP) and 40 μL of accelerator solution (20 μL of DMT in 980 μL of methylene chloride) were added and mixed. To facilitate crosslinking, scaffolds were put into the oven at 40° C. for 1 hour. After crosslinking was completed, the scaffolds were detached from the Teflon holder and the wax was dissolved in a cleaner solution (BIOACT VS-O, Petroferm Inc., Fernandina Beach, Fla.) at 40-60° C. for 1 hour. The scaffolds were dried completely at ambient temperature.

Experimental Setup

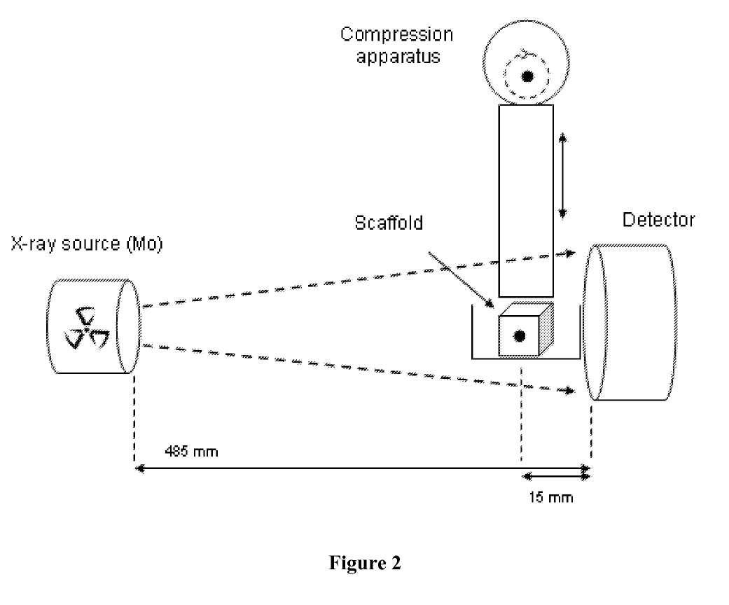

To manufacture the scaffolds, a CAD-based cubic mold (5.0 mm on the side) with a 1.0 mm channel in the middle was generated. After crosslinking, the polymer slightly shrunk and the final scaffold had dimensions 3.1 mm on a side and the channel diameter was 0.56 mm. The scaffold was glued to the bottom of a fluid reservoir placed underneath the loading platen of a custom-made compression device. The setup was mounted inside a custom-made X-ray scanner (FIG. 2). The fluid reservoir was filled with 99.5% glycerin with a viscosity of ˜1.0 Pa s at 25° C., resulting in a slower flow and therefore enabling X-ray exposure times of several seconds. A solution of the radiopaque contrast agent sodium iodide (NaI, Sigma-Aldrich Inc., St. Louis, Mo.) in glycerin (150 mg ml-1) was rendered visible to the eye with 0.5 ml of blue food coloring. A needle syringe with blunt tip (outer diameter of 0.8 mm) was used to infuse the NaI/glycerin solution into the scaffold pore until it was completely filled with contrast agent. The syringe was then slowly withdrawn from the fluid reservoir. Following this infusion, convective transport was induced by cycles of compression and release applied to the top face of the scaffold. The compression amplitude was set at ˜15% of the scaffold height by adjusting the height of the platform below the loading platen. The compression rate was set at 1.0 Hz by adjusting the voltage driving the compression device. The compression amplitude was measured using X-ray projection images of the scaffold in the uncompressed state and in the maximally compressed state.

Projection X-Ray Imaging

The specimens were scanned in a X-ray system consisting of a spectroscopy X-ray source with a molybdenum anode and zirconium foil filter so that the Kalpha emission radiation (17.5 keV) photons predominate in the emitted X-ray spectrum. The specimen's X-ray image was converted into a light image in a Terbium doped fiber optic glass plate and this image was recorded on a Charge Coupled Device (CCD) array, consisting of 1340×1300 pixels with a 20 μm on-a-side pixel resolution. Specimens were placed at 15 mm from the detector and the distance between X-ray source and detector was 485 mm. X-ray exposure time was 5.0 seconds, a scintillator decay time of 0.5 seconds was allowed, and the maximal shutter operation delay was set at 0.2 seconds. The compression device was switched on and off from outside the lead scanner-housing.

Because an exposure time of several seconds was required to generate adequate images, the cyclic compression was intermittently paused after different numbers of compression cycles to allow imaging of the dye distribution. Taking into account the image time as well as time delays caused by the shutter operation, scintillator decay allowance, and turning on and off of the compression apparatus, the delay between subsequent compression intervals was estimated to be no more than 10 seconds.

Image Analysis

For a given spatial coordinate in the image, the transmitted X-ray intensity I is given by:

$$I = {I}_{0} exp ( - {\sum }_{i} {μ}_{i} {x}_{i} ) \qquad ( 1 )$$

where I₀ is the incident X-ray intensity, μᵢ is the linear attenuation coefficient of material i and xᵢ is the thickness of material i along the X-ray beam. The attenuation was due to the diluted contrast agent in the scaffold channel, the polymer material that the scaffold is made of, and the glycerin in the fluid reservoir. Equation 1 can therefore be written as:

$${μ}_{contrast agent} {x}_{channel} + ( {μ}_{polymer} {x}_{scaffold} + {μ}_{glycerin} {x}_{fluid reservoir} ) = - ln ( \frac{I}{{I}_{0}} ) \qquad ( 2 )$$

The attenuation due to the NaI (μ_{contrast agent}χ_{channel}) can be calculated by subtracting the baseline attenuation (i.e., without NaI present) from the negative logarithm of the measured intensity. In a region of interest (ROI) containing the channel (obtained by manual segmentation), the average attenuation was calculated. Under the assumption that the attenuation coefficient of the NaI/glycerin solution is approximately linear to the NaI concentration, the average NaI concentration in the scaffold channel was calculated relative to the average concentration right after injection of NaI in the channel.

### Results

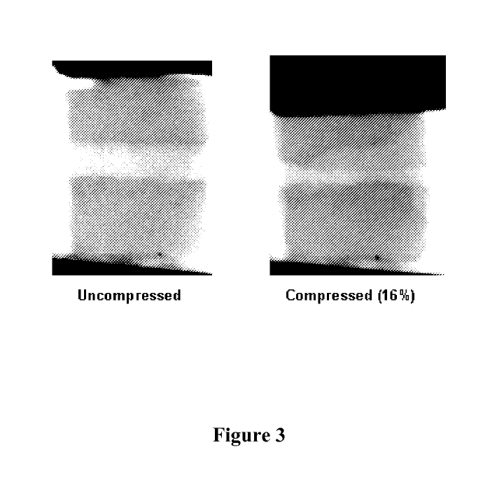

FIG. 3 shows representative projection X-ray images of the scaffold in the uncompressed state compared to the compressed state. The average compression amplitude was 14.5±2.1% of the scaffold height (n=4 experiments). The compression frequency was set at 1.0 Hz.

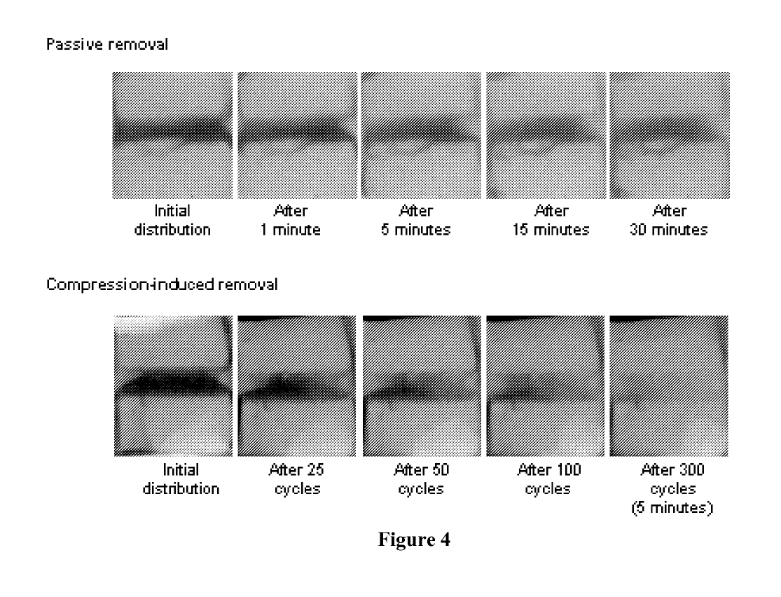

Projection X-ray images with a pixel-resolution of 20 μm show the removal of the contrast agent NaI from the scaffold channel, as caused by passive removal or removal induced by consecutive cycles of deformation of the scaffold (FIG. 4). The images demonstrate that after 30 minutes of passive removal, a substantial amount of the NaI was still present in the channel, whereas most of the NaI was removed from the channel after 300 cycles of compression at 1.0 Hz (i.e., corresponding to 5 minutes). The difference in density between the solution of NaI in glycerin inside the scaffold channel and the glycerin in the fluid reservoir can induce gravitational settling of the dye, and can explain the somewhat higher passive removal rate as would have been expected based on passive transport alone. The gravitational settling is relatively small compared to the compression-induced transport and is inhibited by the higher viscosity of glycerin compared to e.g., water, however.

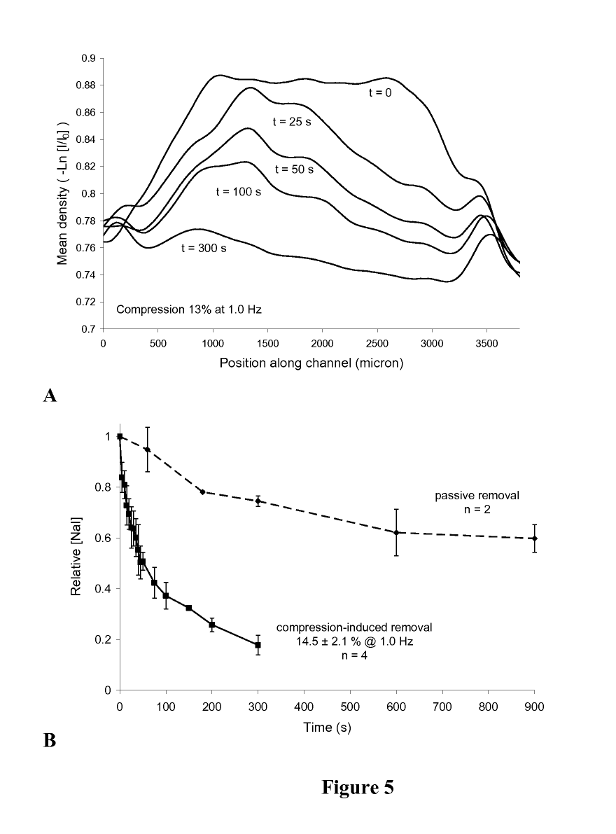

The channel boundaries were defined by manual segmentation and the average NaI concentration inside the channel was calculated as the average attenuation due to iodide. The average NaI concentration was normalized with respect to the NaI concentration after injection. Spatial profiles of the fraction of NaI left in the channel after 0, 25, 50, 100 and 300 cycles of compression (FIG. 5a) demonstrate that the indicator is quickly removed near the channel openings, as compared to locations deeper inside the channel. The results further show the increased removal rate during compression cycles as compared to passive transport alone (FIG. 5b). While passive transport decreased the NaI concentration by only 40% after 60 minutes, the NaI concentration in the channel reached approximately 18% of its initial concentration after 300 compression cycles (corresponding to 5 minutes). To determine the rate constants of NaI removal from the channel, the following function was fitted to the experimentally obtained time course of the compression-induced removal of NaI:

y=A exp(−k₁t)+(1−A)exp(−k₂t) (3)

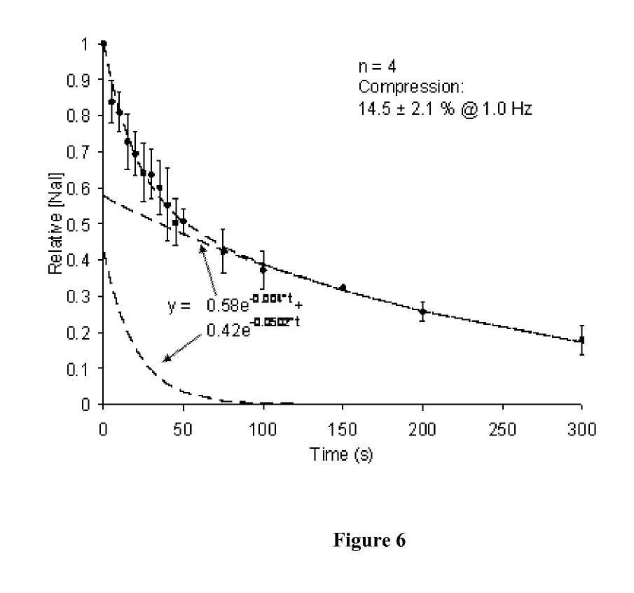

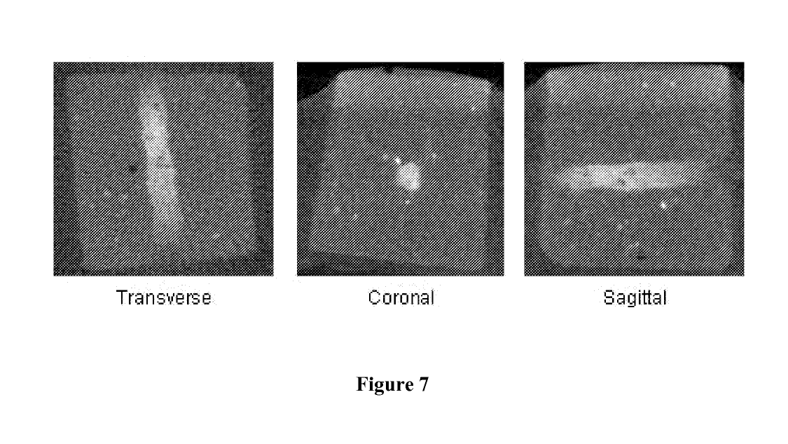

This function with two rate constants k₁ and k₂ could be well fitted to the data (FIG. 6). The following parameter values were obtained: A=0.58, k₁=0.004/second, and k₂=0.0502/second. The individual exponential functions are plotted for comparison, indicating the presence of a slow and a fast rate constant. 3D micro-CT was used to demonstrate three-dimensional imaging of the distribution of contrast agent inside a scaffold flow channel after 50 cycles of compression (FIG. 7).

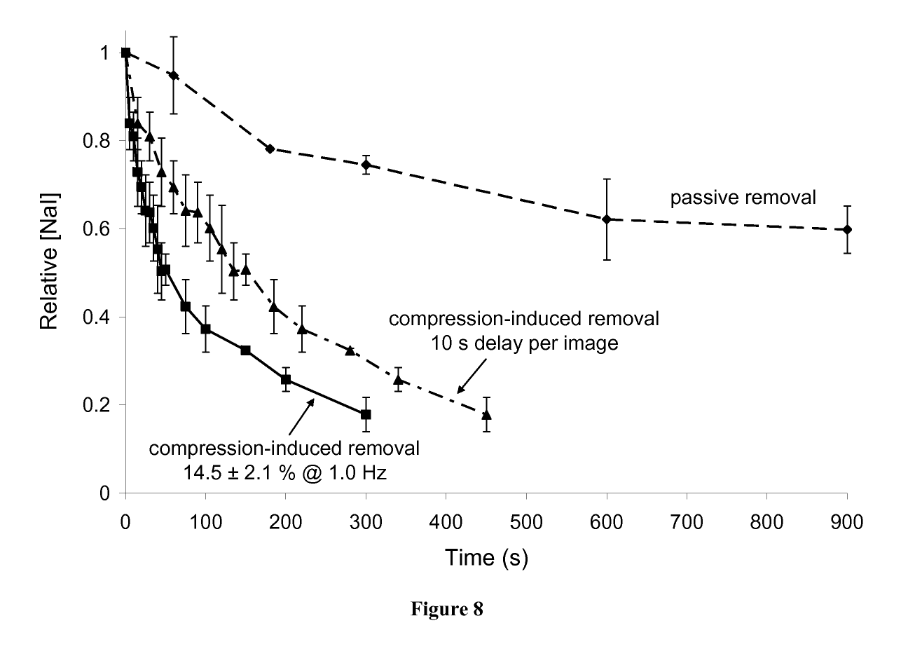

A delay of 10 seconds was added to each measurement point in the time-curve of the compression-induced transport to account for the imaging time (FIG. 8). Including the delay shifted the curve slightly to the right. However, the difference with passive removal is still large.

### CONCLUSION

In this study, dynamic transport of an X-ray contrast agent inside a cyclically deforming porous scaffold was imaged using high-resolution projection X-ray imaging. The current experiment models the transport of nutrients and/or oxygen inside the pore system of a dynamically compressed tissue scaffold. The X-ray imaging methodology described herein offers a means for the experimental validation of theoretical predictions. Using high-resolution X-ray imaging, the pore geometry and quantify local solute concentrations were visualized. In addition, transient alterations in the concentration profile during mechanical deformation of the scaffold were imaged.

### Example 2

### Solute Transport in Deforming Porous Tissue Scaffolds

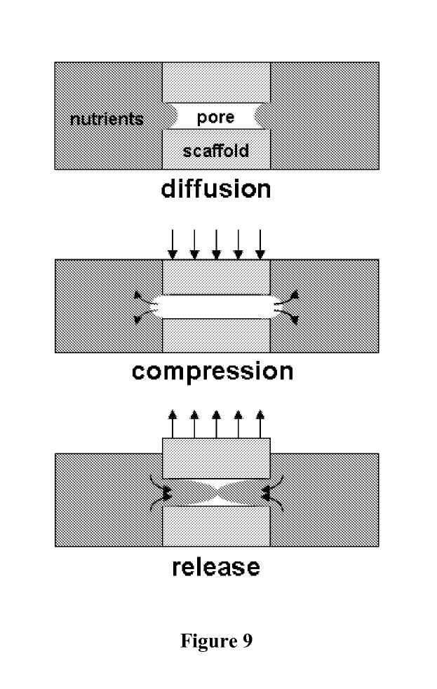

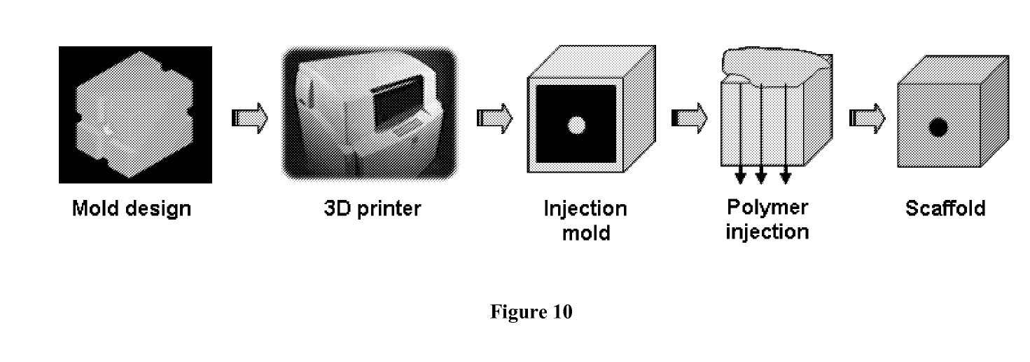

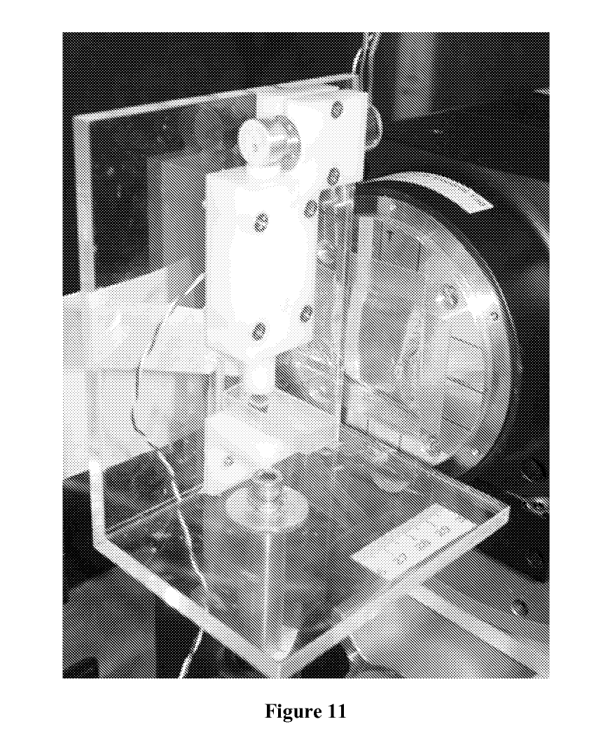

The influence of pore geometry on solute transport in tissue scaffolds during cycles of mechanical compression and release was investigated by means of experimental data and numerical modeling. (FIG. 9) Scaffolds with controllable pore geometries were fabricated with a solid freeform fabrication (SFF) technique as described above (FIG. 10) The distribution of an X-ray tracer inside the scaffolds was imaged after cycles of compression and release using a custom-made high-resolution X-ray system, generating images with a resolution of 20 μm (FIGS. 1 and 11).

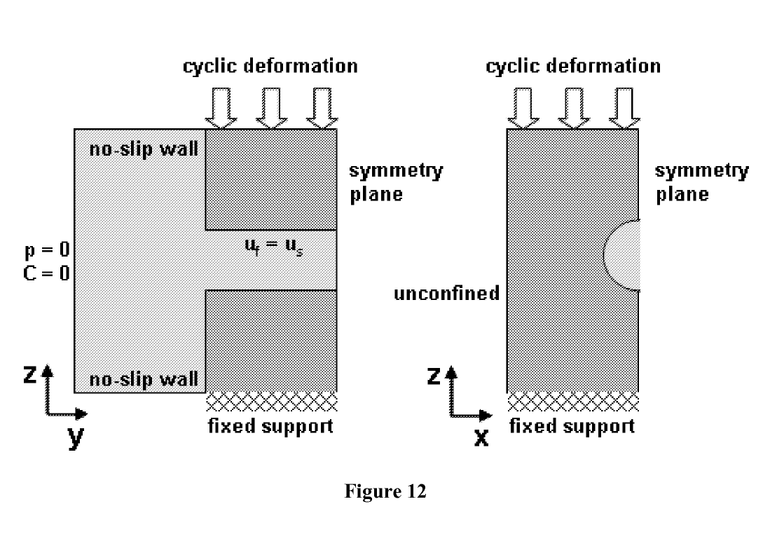

Using the boundary conditions as shown in FIG. 12, a numerical Fluid-Structure Interaction model was compared with empirical data. The model equations are partial differential equations, describing the physical problem in temporal and 3D spatial dimensions, where t is time and x, y and z are the spatial coordinates.

To describe the deformation of the scaffold, a dynamic small-strain problem was considered with a linear Hooke's law as constitutive equation. The following equation describing change in momentum for the scaffold material was used:

$$\frac{\partial {σ}_{x}}{\partial x} + \frac{\partial {τ}_{xy}}{\partial y} + \frac{\partial {τ}_{xz}}{\partial z} = {ρ}_{s} \frac{{\partial }^{2} {u}_{s}}{\partial {t}^{2}} \\ \frac{\partial {σ}_{y}}{\partial y} + \frac{\partial {τ}_{xy}}{\partial x} + \frac{\partial {τ}_{yz}}{\partial z} = {ρ}_{s} \frac{{\partial }^{2} {v}_{s}}{\partial {t}^{2}} \\ \frac{\partial {σ}_{z}}{\partial z} + \frac{\partial {τ}_{xz}}{\partial x} + \frac{\partial {τ}_{yz}}{\partial y} = {ρ}_{s} \frac{{\partial }^{2} {w}_{s}}{\partial {t}^{2}} \qquad ( 4 )$$

where σ_{y}, σ_{y} and σ_{z} are the normal stresses, and τ_{xy}, τ_{xz} and τ_{yz} are the shear stresses. Furthermore, ρₛ is the density of the scaffold material, and uₛ, vₛ and wₛ are the displacements of a material point in the deforming scaffold. Small deformations were considered using a linear strain-displacement relationship:

$$\begin{matrix}{ɛ}_{x} = \frac{\partial {u}_{s}}{\partial x} & {γ}_{xy} = \frac{\partial {v}_{s}}{\partial x} + \frac{\partial {u}_{s}}{\partial y} \\ {ɛ}_{y} = \frac{\partial {v}_{s}}{\partial y} & {γ}_{xy} = \frac{\partial {w}_{s}}{\partial x} + \frac{\partial {u}_{s}}{\partial z} \\ {ɛ}_{z} = \frac{\partial {w}_{s}}{\partial z} & {γ}_{yz} = \frac{\partial {w}_{s}}{\partial y} + \frac{\partial {v}_{s}}{\partial z}\end{matrix} \qquad ( 5 )$$

Here εₓ, ε_{y} and ε_{z} are the normal strains, and γ_{x,y}, γ_{xz} and γ_{yz} are the engineering shear strains. Assuming linear, isotropic elastic behavior, the relation between stress and strain in the scaffold is described by Hooke's law:

$$[ \begin{matrix}{σ}_{x} \\ {σ}_{y} \\ {σ}_{z} \\ {τ}_{yz} \\ {τ}_{xz} \\ {τ}_{xy}\end{matrix} ] = \frac{E}{( 1 + v ) ( 1 - 2 v )} [ \begin{matrix}1 - v & v & v & 0 & 0 & 0 \\ v & 1 - v & v & 0 & 0 & 0 \\ v & v & 1 - v & 0 & 0 & 0 \\ 0 & 0 & 0 & \frac{1}{2} - v & 0 & 0 \\ 0 & 0 & 0 & 0 & \frac{1}{2} - v & 0 \\ 0 & 0 & 0 & 0 & 0 & \frac{1}{2} - v\end{matrix} ] [ \begin{matrix}{ɛ}_{x} \\ {ɛ}_{y} \\ {ɛ}_{z} \\ {γ}_{yz} \\ {γ}_{xz} \\ {γ}_{xy}\end{matrix} ] \qquad ( 6 )$$

where E is the Young's modulus of the scaffold material and v is the Poisson's ratio of the scaffold material. Fluid pressure and velocity of the fluid phase are described by the Navier-Stokes equations. The continuity equation is given by:

$$\frac{\partial {ρ}_{f} }{\partial t} + \frac{\partial ( {ρ}_{f} {u}_{f} )}{\partial x} + \frac{\partial ( {ρ}_{f} {v}_{f} )}{\partial x} + \frac{\partial ( {ρ}_{f} {w}_{f} )}{\partial z} = 0 , \qquad ( 7 )$$

where ρ_{f} is the fluid density, and u_{f}, v_{f} and w_{f} are the fluid velocity components. Density differences in time and space could occur due to mixing of the fluid with the X-ray contrast agent. Laminar flow of a Newtonian fluid with viscosity μ was considered. The following momentum balance of the Navier-Stokes equation was used:

$$\frac{\partial ( {ρ}_{f} {u}_{f} )}{\partial t} + {u}_{f} \frac{\partial ( {ρ}_{f} {u}_{f} )}{\partial x} + {v}_{f} \frac{\partial ( {ρ}_{f} {u}_{f} )}{\partial y} + {w}_{f} \frac{\partial ( {ρ}_{f} {u}_{f} )}{\partial z} = - \frac{\partial p}{\partial x} + μ ( \frac{{\partial }^{2} {u}_{f}}{\partial {x}^{2}} + \frac{{\partial }^{2} {u}_{f}}{\partial {y}^{2}} + \frac{{\partial }^{2} {u}_{f}}{\partial {z}^{2}} ) \\ \frac{\partial ( {ρ}_{f} {v}_{f} )}{\partial t} + {u}_{f} \frac{\partial ( {ρ}_{f} {v}_{f} )}{\partial x} + {v}_{f} \frac{\partial ( {ρ}_{f} {v}_{f} )}{\partial y} + {w}_{f} \frac{\partial ( {ρ}_{f} {v}_{f} )}{\partial z} = - \frac{\partial p}{\partial y} + u ( \frac{{\partial }^{2} {v}_{f}}{\partial {x}^{2}} + \frac{{\partial }^{2} {v}_{f}}{\partial {y}^{2}} + \frac{{\partial }^{2} {v}_{f}}{\partial {z}^{2}} ) \\ \frac{\partial ( {ρ}_{f} {w}_{f} )}{\partial t} + {u}_{f} \frac{\partial ( {ρ}_{f} {w}_{f} )}{\partial x} + {v}_{f} \frac{\partial ( {ρ}_{f} {w}_{f} )}{\partial y} + {w}_{f} \frac{\partial ( {ρ}_{f} {w}_{f} )}{\partial z} = - \frac{\partial p}{\partial z} + μ ( \frac{{\partial }^{2} {w}_{f}}{\partial {x}^{2}} + \frac{{\partial }^{2} {w}_{f}}{\partial {y}^{2}} + \frac{{\partial }^{2} {w}_{f}}{\partial {z}^{2}} ) + g ( {ρ}_{f} - {ρ}_{f , 0} ) \qquad ( 8 )$$

where p is the fluid pressure, ρ_{f,0} is the density of the fluid without contrast agent and g is the gravitation constant.

Solute transport was modeled by the scalar convection-diffusion equation:

$$\frac{\partial C}{\partial t} + \frac{\partial ( {u}_{f} C )}{\partial x} + \frac{\partial ( {v}_{f} C )}{\partial y} + \frac{\partial ( {w}_{f} C )}{\partial z} = D ( \frac{{\partial }^{2} C}{\partial {x}^{2}} + \frac{{\partial }^{2} C}{\partial {y}^{2}} + \frac{{\partial }^{2} C}{\partial {z}^{2}} ) , \qquad ( 9 )$$

where C is the concentration of X-ray contrast agent in the fluid and D the diffusion coefficient. The fluid density is dependent on the concentration of contrast agent:

ρ_{f}=ρ_{f,0}+C (10)

The commercial software ANSYS Workbench 11.0 (Ansys Inc., Canonsburg, Pa.) was used to obtain numerical solutions.

### Results

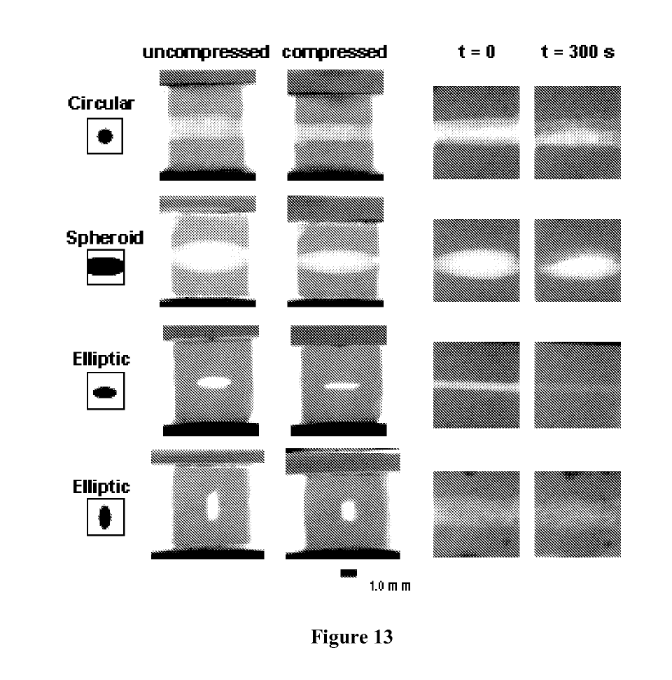

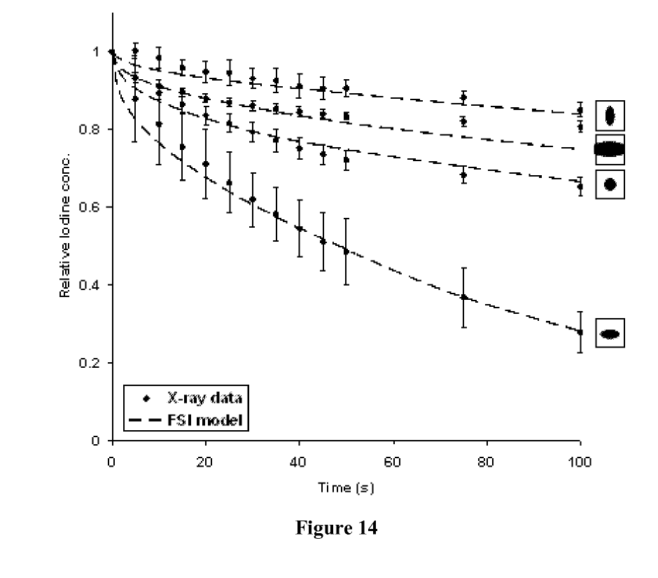

FIGS. 13 and 14 show that solute transport rate and depth in deformable scaffolds are affected by pore shape, size, and cross-sectional orientation with respect to strain direction. FIG. 13 shows the behavior of single flow channels in representative projection X-ray images of the scaffolds in the uncompressed state (first column) compared to the compressed state (second column). The spheroid is mainly compressed in the middle, whereas its openings to the reservoir are slightly narrowed during compression. When the elliptic channel specimen is viewed from the front, the major axis of the elliptic cross-section is perpendicular to the direction of compression. The elliptic cross-section becomes more circular when its major axis is in the same direction as the compression. Projection X-ray images of the initial contrast agent distribution (FIG. 13, third column) and the distribution after 300 compression cycles at ˜10% deformation and 1.0 Hz (last column) show the removal of the contrast agent from the scaffold channel. After 300 cycles of compression, most of the contrast agent is removed from the scaffold with the elliptical cross section pore with semi-minor axis in the main strain direction. On the other hand, only approximately 50% of contrast agent is removed from the circular pore, and most of the dye remains inside the spheroidal pore and the pore with elliptical cross-section with semi-major axis in the main strain direction. FIG. 14 shows the average contrast agent concentration inside the channel as a function of time relative to the concentration at t=0 and shows the good agreement between numerical model to empirical data This allows the Fluid-Structure Interaction Model to be used as an efficient tool for scaffold pore design (FIG. 12).

### Example 3

### Solute Transport in Cyclically Deformed Porous Tissue Scaffolds with Controlled Pore Cross-sectional Geometries

### Introduction

Tissue engineering frequently utilizes porous scaffolds. One use of scaffolds is to cultivate cells on the scaffold in vitro and subsequently implant the construct in vivo. Prior to implantation, bioreactors may be used to perfuse the engineered tissue as to provide cells beyond the diffusion distance with the essential oxygen and nutrients, and to remove toxic waste as a result of cell metabolism and scaffold degradation. Mygind et al., Biomaterials 28: 1036-1047 (2007); Carrier et al., Tissue Eng 8: 175-188 (2002). A functional failure of the implant can occur due to chemotaxis and/or necrosis of cells beyond the diffusion distance. For example, as lack of proper mass transport beyond the diffusional distance after application in vivo can decrease cell density from the periphery to the center of the construct. See, e.g., Karande et al., Ann. Biomed. Eng., 32: 1728-1743 (2004); Ramrattan et al., Tissue Eng., 11: 1212-1223 (2005); and, Silva et al., Biomaterials, 27: 5909-5917 (2006). Where acelluar scaffolds are implanted, infiltration of cells and microvessels into deep layers of the scaffold depends on adequate rate of solute transport. Under static conditions, nutrient transfer is governed by diffusion, i.e. transport driven by a concentration gradient. Diffusive transport is relatively slow (e.g., Fermor et al., Eur. Cell. Mater., 13: 56-65 (2007), reported diffusivities of approximately 2.5·×10⁻⁷ cm²/s for uncharged dextrans in the surface zone of cartilage) and generally accounts for supplying cells at a depth of only a few hundred micrometers from the surface within a reasonable time. See, e.g., Brown et al., Biotechnol. Bioeng., 97: 962-975 (2007).

Solute transport induced by mechanical compression in cubic imaging phantoms with a range of selected pore geometries, representing simplified tissue engineering scaffolds was quantified. Deformable, biodegradable specimens with programmable pore cross-sectional shapes were fabricated using a 3D printing and injection molding technique as described in Examples 1 and 2. The imaging phantoms were immersed in fluid, loaded with an X-ray absorbing dye, and mechanically compressed inside a custom-made X-ray micro scanner. The recorded X-ray images were quantitatively analyzed as to the rate and spatial distribution of solute transport in the porous phantoms.

### Materials and Methods

Scaffold and Pore Geometry

Scaffolds comprising PPF/PCLF were fabricated as described in Example 1. Cubic injection molds (5.0 mm on a side) were printed, such that imaging phantoms with pores consisting of a single channel through the middle of the specimen were generated. Pores with the following cross-sectional and longitudinal shapes were designed: circular cylinder, elliptic cylinder, and spheroid. Five specimens were generated for each shape. After crosslinking of the polymer in the mold, the final dimensions were slightly altered compared to the original design, presumably due to shrinkage of the polymer. The phantoms were scanned in air (no fluid and/or contrast agent present) with micro-CT at 20 μm voxel resolution using a custom-made X-ray imaging system to determine the dimensions accurately. No swelling or shrinking of the specimens after immersion in fluid was observed.

Experimental Setup

Experiments were carried out at room temperature. The imaging phantoms were glued to the bottom of a fluid reservoir placed underneath the loading platen of a custom-made compression device. The setup was mounted inside a custom-made high-resolution X-ray imager (FIG. 11). The fluid reservoir was filled with 99.5% glycerol with a viscosity of ˜1.0 Pa s, which closely matched the viscosity of the iodine-based contrast medium, decreasing gravitational settling of the tracer and therefore enabling X-ray exposure times of several seconds. A solution of the radiopaque contrast agent sodium iodide (NaI, Sigma-Aldrich Inc., St. Louis, Mo.) in glycerol (25.8 mg/ml I⁻) was rendered visible to the eye with a drop of blue food coloring. The X-ray contrast agent NaI is based on the high atomic weight of the element iodine. In addition, the concentration of NaI was higher than most physiological substances to ensure a reasonable signal-to-noise ratio in the images. This resulted in gravitation-induced convection of the tracer, explaining the somewhat higher passive removal rate as would have been expected based on diffusional transport alone. To reduce the influence of gravitational settling, glycerol with a viscosity of ˜1.0 Pa second (as compared to 1.0 mPa second for water and 1.31 mPa second for human blood plasma as described in Kasser et al., Biorheology 25: 727-741 (1988)) was used as the solvent. A needle syringe with blunt tip was used to infuse the contrast agent into the specimen's pore until it was completely filled, and the syringe was then slowly withdrawn from the fluid reservoir. Convective transport was induced by cycles of compression and release applied to the top face of the specimen. The compression amplitude was measured using X-ray projection images of the specimen in the uncompressed state and in the maximally compressed state. The percentage of compression was calculated from the images as:

$$\vert \frac{H - {H}_{0} }{{H}_{0}} \vert \cdot 100 \% \qquad ( 11 )$$

where H₀ and H are the phantom height at rest and upon maximal compression respectively. The compression rate was set at 1.0 Hz.

Projection X-Ray Imaging Protocol

The specimens were imaged in a custom-made high-resolution X-ray system as described in Example 1. The pixel size in the X-ray image was 20 μm, so that the spatial resolution is approximately 40 μm and, hence, pore diameter differences of ˜500 μm can be resolved. Specimens were placed at 5.5 cm from the detector and the distance between X-ray source and detector was 98.5 cm. X-ray exposure time was 5.0 seconds and a scintillator decay time of 0.5 seconds was allowed for. The compression device was switched on and off from outside the lead-lined scanner-housing. Images of the fluid filled specimens were recorded before and right after injection of the contrast agent. During ‘passive’ experiments (compression turned off), images were recorded after 1, 3, 5 and 10 minutes. During ‘active’ experiments (compression turned on) images were recorded after 5, 10, . . . , 50, 75, 100, 150, 200 and 300 compression cycles, by temporarily pausing the cyclic compression during the imaging time of 5.7 seconds, with the specimen in the uncompressed state. The total imaging time was less than 2 minutes per experiment. The contribution of passive removal was expected to be less than 10% to the total solute transport.

Image Analysis

The transmitted X-ray intensity I at each pixel is given by Equation 1, where I₀ is the incident X-ray intensity, μᵢ is the linear attenuation coefficient of material i and xᵢ is the thickness of material i along the X-ray beam illuminating the pixel after passing through the imaging phantom. The total X-ray attenuation is mainly due to the iodine in the channel, the polymer material that the specimen is made of, and the glycerol in the surrounding fluid reservoir. The attenuation due to iodine in the pore was calculated by subtracting the attenuation before contrast agent injection from the attenuation measured with iodine present in the pore. The average attenuation due to iodine was obtained by averaging the attenuation over the entire pore volume. Under the assumption that the attenuation coefficient of the iodine is linearly proportional to the iodine concentration (because the pore dimension remains unchanged in between compressions), the average iodine concentration in the channel was calculated as a fraction of the average iodine concentration calculated immediately after injection.

Statistical Analysis

To compare the results of the image analysis, the remaining fraction of iodine (as measured after 300 seconds of passive removal or after 300 compression cycles at 1.0 Hz) was evaluated with one-way analysis of variance (ANOVA). This fraction was calculated in specimens with different channel shapes and compared with the 0.5 mm circular cylindrical channel by a Tukey-Kramer honestly significant difference test (#: P<0.05). For each channel shape, the effect of passive and deformation-induced transport was compared by a two-tailed t-test (*: P<0.05).

### Results

Scaffold Pore Geometry

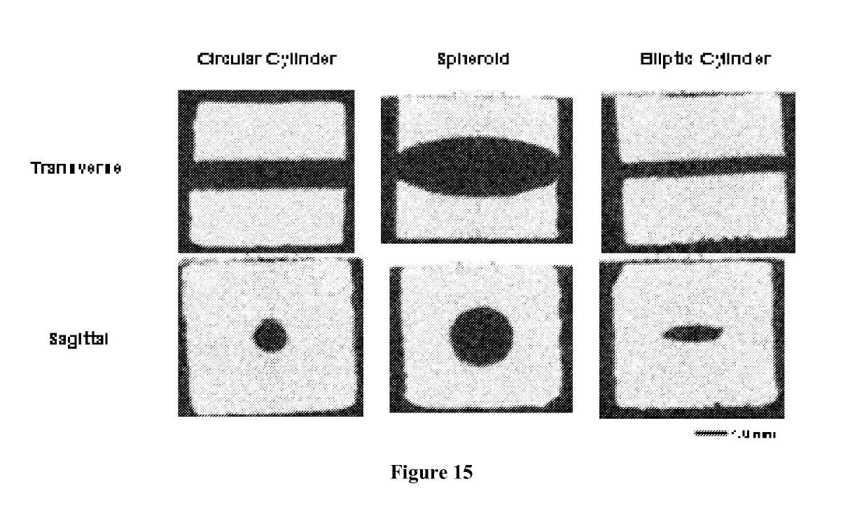

Representative micro-CT images (20 μm voxel resolution) of the specimens are shown in FIG. 15. These images were used to quantify the actual (as distinct from the programmed) pore dimensions (Table 1). The average side dimension of all specimens was 4.50±0.18 mm.

TABLE 1
Channel dimensions of initial computer-aided design (CAD) models and
actual specimens after manufacturing as measured with micro-CT.

| | Circular cylinder channel diameter (mm) | | | |
|---|---|---|---|---|
| CAD design | 0.5 | 1.0 | 1.5 | 2.0 |
| Micro-CT | Z0.37 ± 0.030 | 0.94 ± 0.026 | 1.38 ± 0.048 | 1.89 ± 0.028 |
| measured | | | | |
| | Elliptic cylinder channel | | Spheroid channel | |
| | diameters (mm) | | diameters (mm) | |
| | minor axis | major axis | at openings | Maximum |
| CAD design | 0.6 | 2.0 | 0.55 | 2.0 |
| Micro-CT | 0.54 ± 0.043 | 1.75 ± 0.043 | 0.73 ± 0.076 | 1.89 ± 0.017 |
| measured | | | | |

For the circular cylinder, scaffolds with four different diameters were generated.
For the elliptic cylinder, the minor and major axis dimensions of the channel cross-section are given.
For the spheroid, the diameter at the channel openings, and the maximum diameter in the middle are given.
All specimens were cubic with an average side dimension of 4.50 ± 0.18 mm.

Scaffold Compression

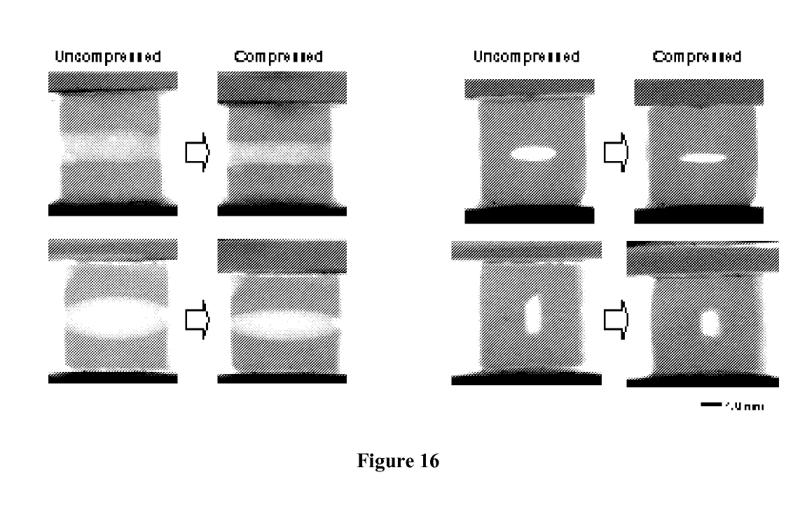

FIG. 16 shows representative projection X-ray images of the scaffold in the uncompressed state compared to the compressed state. The spheroid is mainly compressed in the middle, whereas its openings to the reservoir are slightly narrowed during compression. From the front view of the specimen with elliptic channel, it can be observed that when the major axis of the elliptic cross-section is perpendicular to the direction of compression, it is highly deformed (65±5% compression along the semi-minor axis, see Table 2). In contrast, the elliptic cross-section becomes more circular when its major axis is in the same direction as the compression.

On average, the percentage of scaffold compression was 8.6±1.6% of the original specimen height and not significantly different among the different scaffolds as tested by ANOVA. Thus, the scaffold compression amplitude did not depend on the pore shape. For the circular cylindrical channels with different diameters, the percentage of compression of the channel decreased with increasing diameter (Table 2).

TABLE 2
Percentage compression of the channels

| | CAD diameter | Channel |
|---|---|---|
| Channel geometry | (mm) | compression (%) |
| Circular | 0.5 | 45 ± 6 |
| | 1.0 | 48 ± 4 |
| | 1.5 | 33 ± 3^{#} |
| | 2.0 | 26 ± 1^{#} |
| Elliptic | 0.6 | 65 ± 5^{#} |
| (strain parallel to semi-minor axis) | | |
| Elliptic | 2.0 | 18 ± 1^{#} |
| (strain parallel to semi-major axis) | | |
| Spheroid | 2.0 | 17 ± 1^{#} |

As measured along the vertical direction.
^{#}significantly different from 0.5 mm (P < 0.05).

Solute Transport

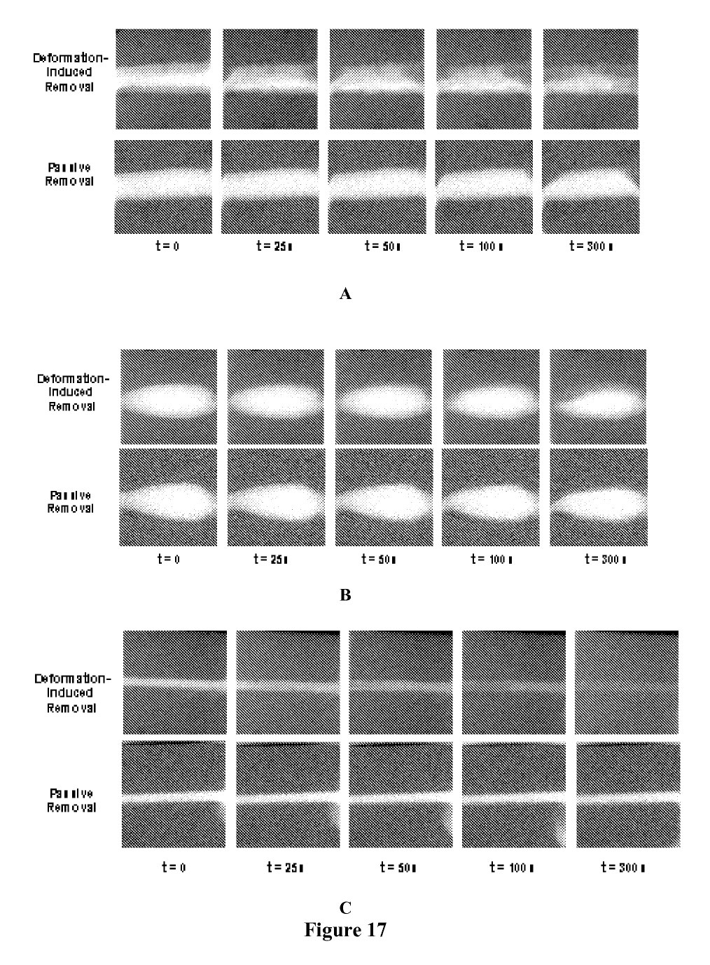

Projection X-ray images show the removal of the iodine from the scaffold channel, as caused by passive removal or removal induced by consecutive cycles of deformation of the scaffold (FIG. 17). The images demonstrate that after 300 seconds of passive removal without compression, most of the iodine is still present in the channels. A portion of the contrast agent is slowly removed, in part due to gravity-induced natural convection because of the slight difference in density between the fluid containing NaI and the fluid without the contrast agent. In addition, after 300 cycles of compression at 1.0 Hz (i.e., corresponding to 300 seconds), most of the contrast agent was removed from the phantom with the elliptical cross section pore. In contrast, only approximately 50% of iodine was removed from the circular pore, and most of the dye remains inside the spheroidal pore.

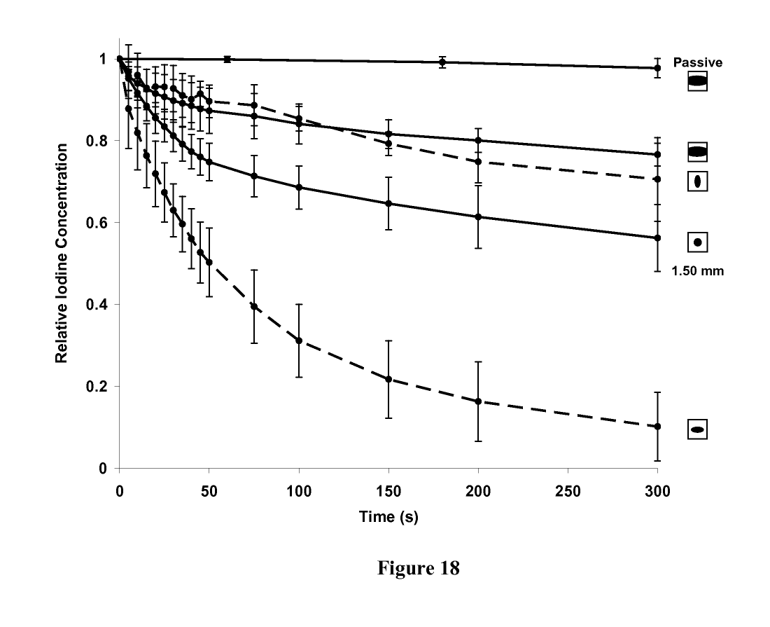

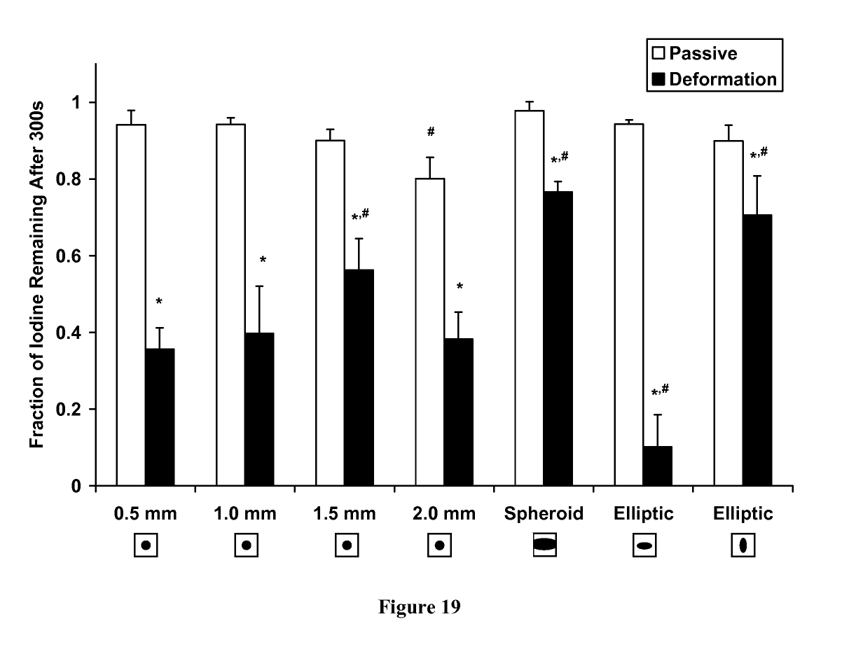

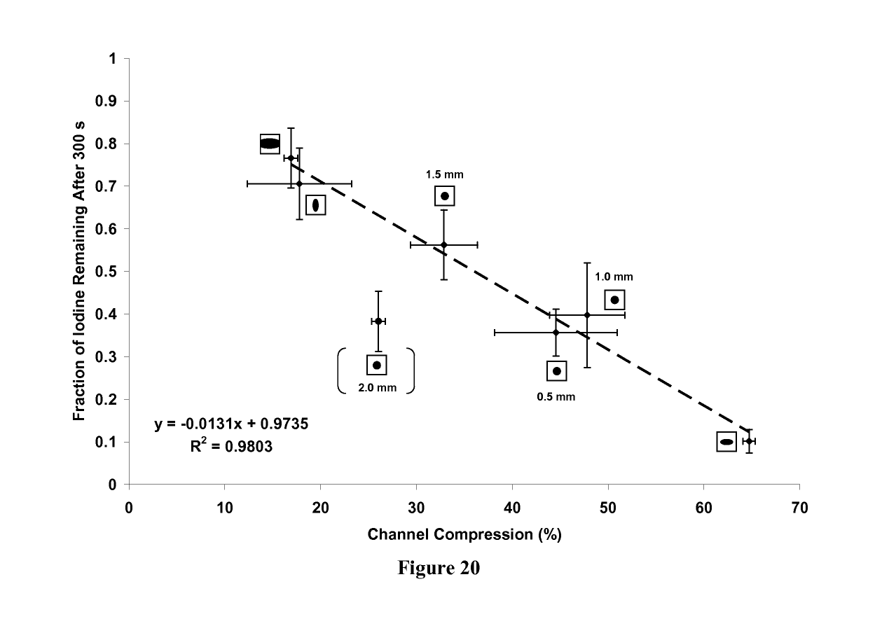

The average iodine concentration inside the channel was calculated relative to the iodine concentration right after injection. This quantitative analysis shows the effect of the pore shape on the removal rate during compression cycles (FIG. 18). The remaining fraction of iodine in the channel after 300 seconds is shown (FIG. 19) for all channel shapes as a result of both passive and deformation-induced removal. A statistically significant difference between passive and strain-induced removal was found for all pore shapes. The remaining fraction of iodine 300 seconds after passive removal was statistically different from the 0.5 mm circular only for the 2.0 mm pore, likely due to increased gravitational settling. After 300 seconds of strain-induced removal, the remaining fraction of iodine was significantly different from the 0.5 mm pore for the 1.5 mm circular pore, the spheroid pore, and the elliptic pore (in both directions of compression). With the exception of the 2.0 mm circular pore, the remaining fraction of iodine in the channel after 300 seconds correlated well with the channel compression (FIG. 20). Upon exclusion of the 2.0 mm circular pore, linear regression yielded a relationship of y=−0.00131 x+0.9735, with R²=0.9803. Increased gravitational settling in the 2.0 mm pore likely caused the increased rate of solute transport, as would be expected based on the percentage of channel compression.

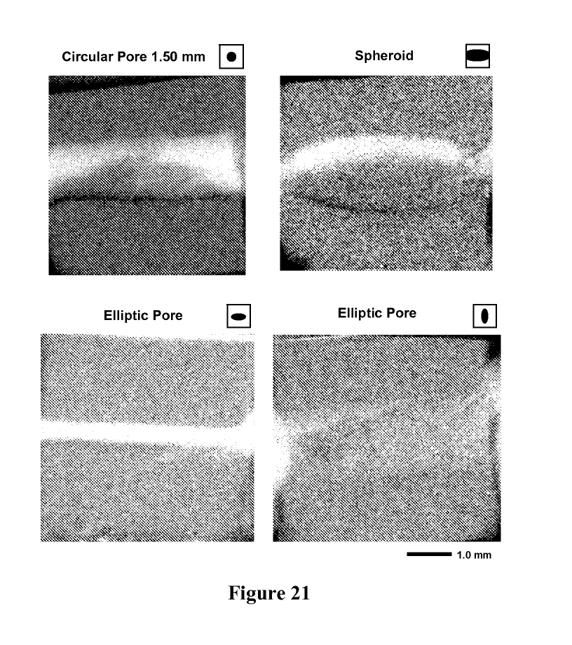

To illustrate the spatial distribution of solute transport in the different scaffold types, images recorded right after iodine injection were subtracted from images recorded after 300 compression cycles (FIG. 21). White areas in these images represent spots where most of the contrast agent has been removed after 5 minutes of cyclic deformation.

Dynamic transport of an X-ray tracer inside cyclically deformed imaging phantoms with designed pore geometries, mimicking porous tissue scaffolds, was imaged using an X-ray micro imaging technique. These results show that solute transport rates and depths can be significantly influenced by the shape of the pore, its dimension, and the orientation of its cross-section with respect to the direction of the cyclic strain. For example, increasing the diameter of the circular cylindrical channels from 0.5 mm to 1.5 mm slightly decreased the deformation induced solute transport rates, which correlated with the decreased percentage of channel compression of the 1.5 mm channel. The increased passive removal in the 2.0 mm diameter channel as compared to the 0.5 mm channel most likely compensated for this effect. The spheroidal channel showed the slowest transport rates during both passive removal and compression-induced removal. This can be attributed to its relatively large volume compared to its smaller cross-section exposed to the surrounding fluid reservoir. The elliptic cylinder with its major axis perpendicular to the direction of compression was highly collapsible and therefore yielded a high solute transport rate under cyclic compression. In contrast, when its major axis was in parallel with the direction of compression, solute transport was significantly reduced, indicating the strong influence of pore orientation compared to direction of strain.

Limited mass transport currently hinders the development of thick tissue-engineered implants and oxygen (O₂) is one of the most important metabolic substrate to be transported to the cells inside the scaffolds to maintain normal cell function. Sensitivity to hypoxia varies among cells: 40% of cells cultured under hypoxia do not survive after ˜5 days for endothelial cells, after ˜12 hours for cardiomyocytes, and after only ˜2 hours for preadipocytes (for adipose tissue engineering). See, e.g., Dore-Duffy et al., Microvasc Res, 57: 75-85 (1999); Mehrhof et al., Circulation, 104: 2088-2094 (2001); and, Patrick et al., Semin. Surg. Oncol., 19: 302-311 (2000). To sustain viable cells inside the scaffolds, O₂ transport rate must match rate of O₂ consumption (e.g., 1 to 10 nmol O₂/min/10⁶ cells as described in Petit et al., Mitochondrion, 5: 154-161 (2005) and Casey et al., Circulation, 102: 3124-3129 (2000). The present results indicate that the time to reach 37% of the initial iodine (as a surrogate for O₂) concentration for the elliptic pore compressed along its semi-minor axis was approximately 1 minute (FIG. 18), yielding an average transport rate of 0.63/minute. Given an arterial plasma O₂ concentration of 130 μmol L−1 and an average O₂ consumption of 5 nmol O₂/minute/10⁶ cells, an estimate for the sustainable cell density in the scaffold can then be calculated:

$$\frac{130 \cdot {10}^{- 6} mol {L}^{- 1} \cdot 0.63 {min}^{- 1}}{5 \cdot {10}^{- 9} mol {min}^{- 1} {( {10}^{6} cells )}^{- 1}} = 1.6 \cdot {10}^{7} cells {ml}^{- 1} \qquad ( 12 )$$

This is still one to two orders of magnitude lower than cell densities in most human vascularized tissues; however, cyclic strain may induce sufficient temporary convective nutrient transport to maintain viable cells while ingrowth of microvessels proceeds after implantation of the scaffold. Even more, although solute convection dominates transport in these experiments, the diffusion coefficient of O₂ in aqueous solution is likely higher as compared to NaI in glycerol due to lower solvent viscosity and lower molecular weight (32 g/mol for O₂ vs. 149.9 g/mol for NaI), which could increase the sustainable cell density.

Increased convective transport properties of the scaffold will trade off with its ability to provide temporary mechanical support at the site of implantation. Pores with elliptical cross-section oriented with the semi-minor axis along the strain direction can yield high transport rates, but the effective scaffold stiffness will be lower than when a pore with e.g., a circular cross-section is used. The solid freeform technique used in this study allows the fabrication of scaffolds with programmable pore labyrinths.

Although pore diameters of the imaging phantoms ranged from 370 μm to 1.91 mm, these results can be relevant for pores with smaller dimensions. ‘Optimal’ pore sizes for tissue engineering scaffolds have been suggested to lie between 100 and 500 μm, depending on the cell type. See, e.g., Ikada et al., J. R. Soc. Interface, 3: 589-601 (2006). Bone ingrowth has been demonstrated in scaffolds with pore sizes greater than 1.0 mm as manufactured by SFF or a combination of phase-inversion and particulate extraction as described in Hollister et al., Orthod. Craniofac. Res., 8: 162-173 (2005) and Holy et al., J Biomed Mater Res, A65: 447-453 (2003).

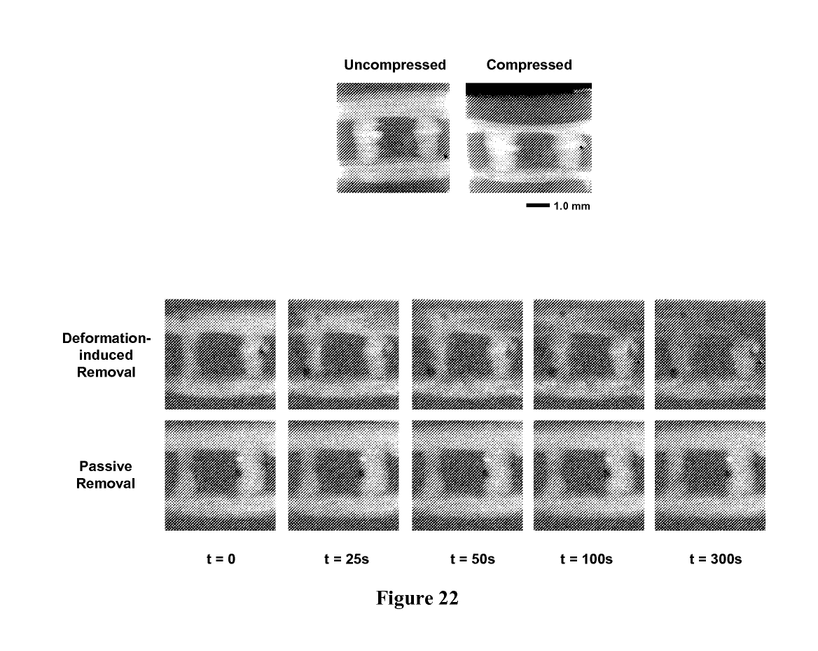

‘Scaffolds’ with simple pores comprised of single straight channels with various shapes were used, whereas more realistic scaffolds would have pore labyrinths comprised of interconnected channels in three dimensions. Using the described scaffold fabrication technique, more complex pore structures can be manufactured. FIG. 22 shows a qualitative demonstration of the techniques described in this paper, applied to a simple pore network of 1.0 mm diameter interconnected circular channels in a single plane.

The results demonstrate that shape, size, and orientation of pores in a tissue scaffold have great effects on solute transport during cyclic mechanical deformation. This has implications for the design of the pore system of thick, deformable implants in which enhanced solute transport rates are desired to facilitate tissue ingrowth. Additionally, pore geometry may be adjusted to achieve ideal release constants in deformable porous drug delivery systems.

### Example 4

### Imaging Experiments with Phantoms

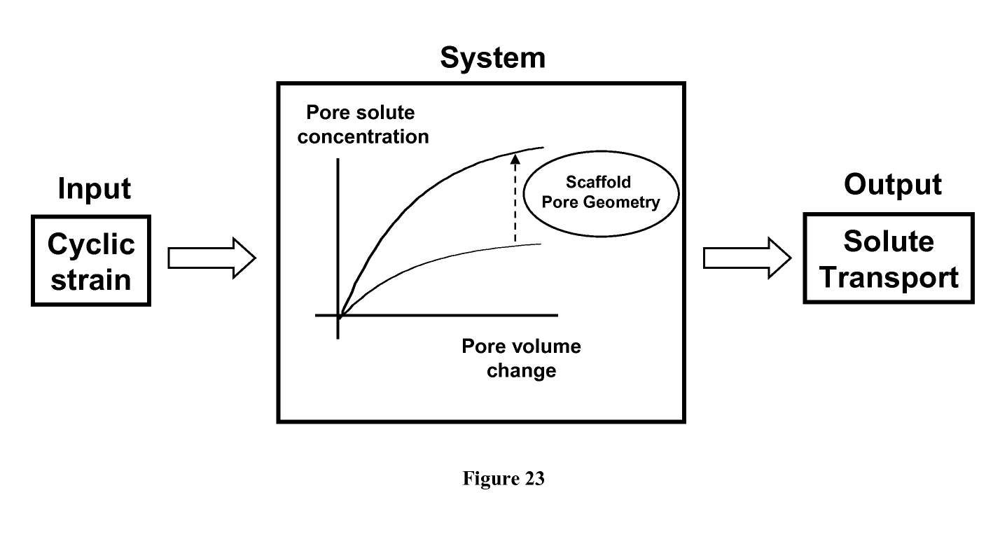

Imaging experiments with phantoms representing simple tissue scaffolds were conducted to investigate the influence of pore cross-sectional geometry, flow channel diameter, pore cross-sectional alignment with respect to the main strain direction and the presence of interconnections on the rate of outward transport of a tracer (as a surrogate for waste products). Flexible cubic scaffolds with side dimensions of ˜5 mm were generated with a range of programmed pore geometries using a combined 3D printing and injection molding technique. The scaffolds were cyclically compressed with amplitudes of ˜10-15% of the scaffold height at 1.0 Hz. The (unconfined) compression was applied at the top face of the scaffold while the scaffold was immersed in a fluid reservoir. The pores of the scaffold were loaded with a contrast agent for X-ray or optical contrast, and the removal of the contrast agent as a result of cyclic pumping was quantified. The assumption is made that outward transport (e.g., by waste products) is the opposite of inward transport (e.g., by nutrients and oxygen). These data suggest that scaffold pore geometry can be modified to control the solute transport rate induced by cyclic loading of the scaffold (FIG. 23).

Effect of Interconnections Between Pores

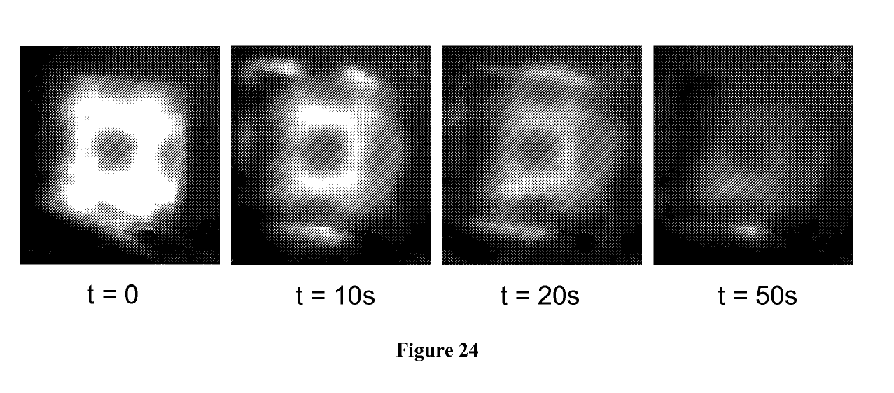

FIG. 24 shows representative images of the tracer distribution (white) in a scaffold with interconnected pores with circular cross-section. The pores are interconnected in a plane and the cyclic compression was applied in the normal direction of that plane (i.e., the images were taken from the bottom looking upwards). It can be seen that the tracer is rapidly washed out as a result of the lower hydraulic resistance due to channel interconnections.

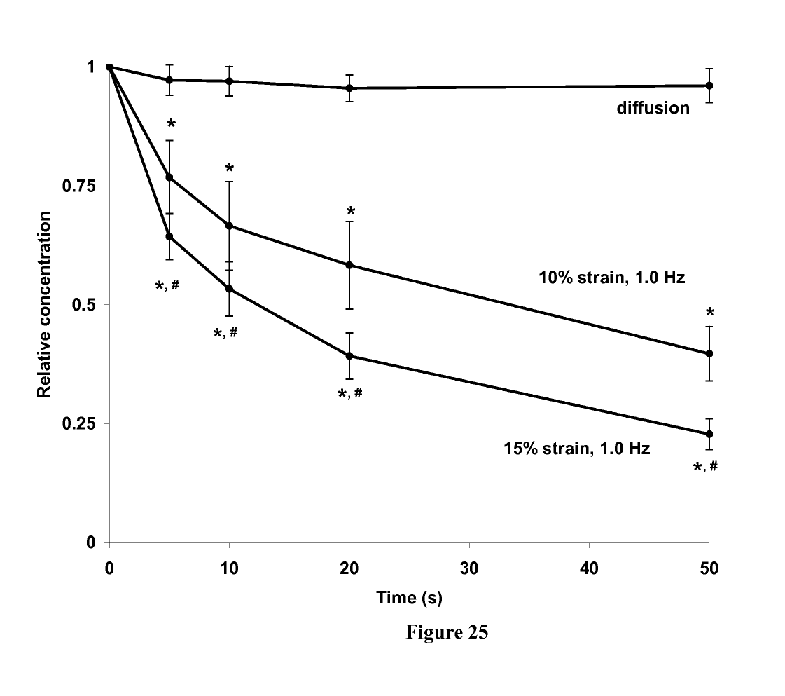

FIG. 25 shows the quantified average tracer concentration relative to the concentration at t=0 for the scaffold with interconnected circular flow channels. The scaffolds were cyclically compressed with an amplitude of 0% (i.e., diffusion), 10% and 15% of the original scaffold height, at a frequency of 1.0 Hz. The removal rate in a scaffold with interconnected channels is higher than in a single channel scaffold due to the overlap in volume and reduced resistance to flow. Thus, interconnections can enhance solute transport in porous tissue scaffolds.

Possibility of Directional Flow

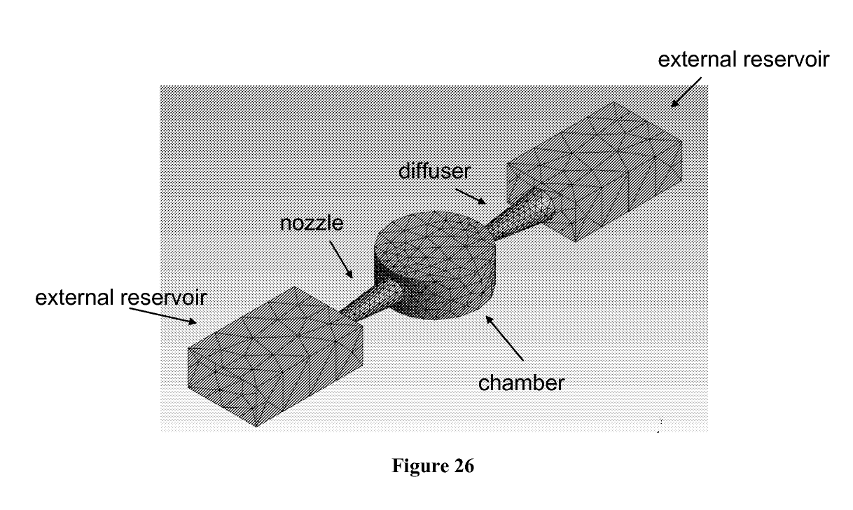

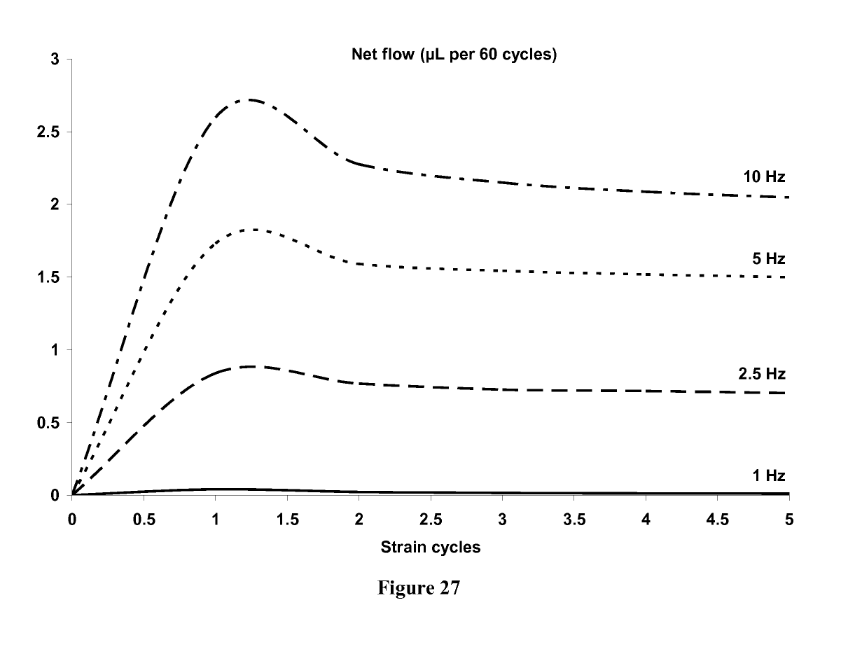

Investigations with computational fluid dynamics towards the possibility to induce a net fluid flow through the scaffold upon cyclic compression were performed. The concept consists of a pumping chamber connected to two nozzle-diffuser type channels (FIG. 26). The nozzle-diffuser type elements are conical channels with angles of approximately 5 to 10°. These channels have hydraulic resistances that are dependent on the flow-direction (resistance is higher in the converging direction than in the diverging direction) due to which the fluid is preferentially pumped into the diverging direction of the channels and our computations suggest that the net flow is frequency dependent (FIG. 27). Such scaffold designs can be manufactured relatively easily with a solid freeform fabrication technology.

### Example 5

### Validation of a Fluid-Structure Interaction Model of Solute Transport in Pores of Cyclically Deformed Tissue Scaffolds

Experiments were conducted to develop a computational model of deformation-induced solute transport in porous tissue scaffolds, which included the pore geometry of the scaffold. This geometry consisted of a cubic scaffold with single channel in the middle of the scaffold, immersed in a fluid reservoir.

X-Ray Experiments

Experiments were described in Op Den Buijs et al., (Tissue Eng Part A 15:1989-99, (2009)). In brief, flexible cubic scaffolds were fabricated from a biodegradable polymer blend (75% polycaprolactone fumarate and 25% polypropylene fumarate) using a combined 3D printing and injection molding technique. Cubic injection molds were printed, such that scaffolds with pores consisting of a single channel through the middle of the specimen could be generated. Pores with the following cross-sectional and longitudinal shapes were designed: circular cylinder, elliptic cylinder and spheroid (5 specimens per shape). After fabrication, the scaffolds were scanned with micro-CT at 20 μm isotropic voxel resolution, to obtain their final dimensions. The imaging phantoms were attached to the bottom of a fluid reservoir placed underneath the loading platen of a custom-made compression device. The specimens were loaded with a solution of the radiopaque solute sodium iodide dissolved in glycerin (31 mg ml⁻¹). The solute distribution was quantified by recording 20 μm pixel-resolution images in an X-ray micro-imaging scanner at selected time points after intervals of dynamic straining with a mean strain of 8.6±1.6% at 1.0 Hz.

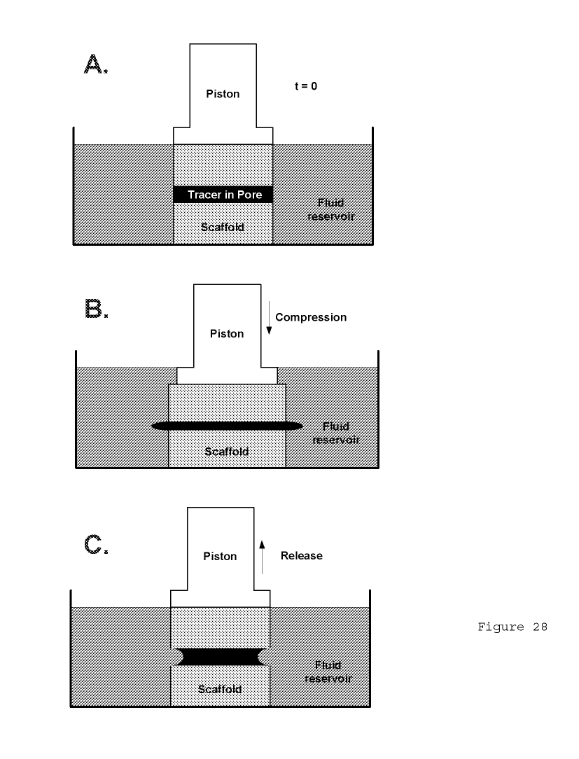

One aim of the computational model was to represent the experiment depicted in FIG. 28. The model domain consisted of a solid phase (i.e., the scaffold material) and a fluid phase (i.e., the fluid inside the pore system of the scaffold and the surrounding fluid reservoir). The pore system of the scaffold was initially filled with a fluid containing the X-ray tracer (used in the experiments a surrogate for nutrients and waste products). The scaffold was then cyclically compressed, resulting in a deformation of the scaffold and movement of the fluid inside the scaffold: fluid flows out of the pores upon compression and back into the pores upon release of the scaffold. This ‘pumping effect’ inside the scaffold aimed to augment removal of the X-ray contrast agent inside the scaffold as compared to diffusive transport alone. The model did not take into account the presence of cells in the pores that deplete nutrients and may influence fluid flow. Any changes in the pore geometry due to biodegradability of the scaffold material were neglected. Furthermore, it was assumed that the scaffold material did not absorb any of the solute, i.e. mass transport was only described in the pores of the scaffolds.

Model Equations

The model equations are partial differential equations, describing the physical problem in temporal and 3D spatial dimensions, where t is time and x, y and z are the spatial coordinates.

Scaffold Deformation

To describe the deformation of the scaffold, a dynamic small-strain problem was considered with a linear Hooke's law as constitutive equation. Neglecting external body forces on the scaffold material, the equation describing change in momentum for the scaffold material was given by

$$\begin{matrix}\frac{\partial {σ}_{x}}{\partial x} + \frac{\partial {τ}_{xy} }{\partial y} + \frac{\partial {τ}_{xz}}{\partial z} = {ρ}_{s} \frac{{\partial }^{2} {u}_{s}}{\partial {t}^{2}} \\ \frac{\partial {σ}_{y}}{\partial y} + \frac{\partial {τ}_{xy}}{\partial x} + \frac{\partial {τ}_{yz}}{\partial z} = {ρ}_{s} \frac{{\partial }^{2} {v}_{s}}{\partial {t}^{2}} & ( 13 ) \\ \frac{\partial {σ}_{z}}{\partial z} + \frac{\partial {τ}_{xz}}{\partial x} + \frac{\partial {τ}_{yz}}{\partial y} = {ρ}_{s} \frac{{\partial }^{2} {w}_{s}}{\partial {t}^{2}} , & \end{matrix}$$

where σₓ, σ_{y} and σ_{z} are the normal stresses, and τ_{xy}, τ_{xz} and τ_{yz} are the shear stresses. Furthermore, ρₛ is the density of the scaffold material, and uₛ, vₛ and wₛ are the displacements of a material point in the deforming scaffold. Small deformations were considered, and the relation between the strains and the displacements using the linear relationships was described as

$${ɛ}_{x} = \frac{\partial {u}_{s}}{\partial x} \\ {γ}_{xy} = \frac{\partial {v}_{s}}{\partial x} + \frac{\partial {u}_{s}}{\partial y} \\ {ɛ}_{y} = \frac{\partial {v}_{s}}{\partial y} \\ {γ}_{xz} = \frac{\partial {w}_{s}}{\partial x} + \frac{\partial {u}_{s}}{\partial z} \\ {ɛ}_{z} = \frac{\partial {w}_{s}}{\partial z} \\ {γ}_{yz} = \frac{\partial {w}_{s}}{\partial y} + \frac{\partial {v}_{s}}{\partial z} \qquad ( 14 )$$

Here εₓ, ε_{y} and ε_{z} are the normal strains, and γ_{xy}, γ_{xz} and γ_{y}, are the engineering shear strains. Assuming linear, isotropic elastic behavior, the relation between stress and strain in the scaffold was described by Hooke's law:

$$[ \begin{matrix}{σ}_{x} \\ {σ}_{y} \\ {σ}_{z} \\ {τ}_{yz} \\ {τ}_{xz } \\ {τ}_{xy }\end{matrix} ] = \frac{E}{( 1 + v ) ( 1 - 2 v )} [ \begin{matrix}1 - v & v & v & 0 & 0 & 0 \\ v & 1 - v & v & 0 & 0 & 0 \\ v & v & 1 - v & 0 & 0 & 0 \\ 0 & 0 & 0 & \frac{1}{2} - v & 0 & 0 \\ 0 & 0 & 0 & 0 & \frac{1}{2} - v & 0 \\ 0 & 0 & 0 & 0 & 0 & \frac{1}{2} - v\end{matrix} ] [ \begin{matrix}{ɛ}_{x} \\ {ɛ}_{y} \\ {ɛ}_{z} \\ {γ}_{yz} \\ {γ}_{xz } \\ {γ}_{xy}\end{matrix} ] \qquad ( 15 )$$

where E is the Young's modulus and v is the Poisson's ratio of the scaffold material.

Fluid Motion

Fluid pressure and velocity of the fluid phase were described by the Navier-Stokes equations. The continuity equation was given by:

$$\frac{\partial {ρ}_{f}}{\partial t} + \frac{\partial ( {ρ}_{f} {u}_{f} )}{\partial x} + \frac{\partial ( {ρ}_{f} {v}_{f} )}{\partial y} + \frac{\partial ( {ρ}_{f} {w}_{f} )}{\partial z} = 0 , \qquad ( 16 )$$

where ρ_{f} is the fluid density, and u_{f}, v_{f} and w_{f} are the fluid velocity components. It should be noted that, although the fluid was assumed to be incompressible, density differences in time and space could occur due to mixing of the fluid with the X-ray contrast agent. Furthermore, laminar flow of a Newtonian fluid with viscosity μ was described including a buoyancy source term to model density differences due to mixing with the X-ray absorbing contrast agent, which has a higher density. The momentum balance of the Navier-Stokes equation was given by:

$$\frac{\partial ( {ρ}_{f} {u}_{f} )}{\partial t} + {u}_{f} \frac{\partial ( {ρ}_{f} {u}_{f} )}{\partial x} + {v}_{f} \frac{\partial ( {ρ}_{f} {u}_{f} )}{\partial y} + {w}_{f} \frac{\partial ( {ρ}_{f} {u}_{f} )}{\partial z} = - \frac{\partial p}{\partial x} + μ ( \frac{{\partial }^{2} {u}_{f}}{\partial {x}^{2}} + \frac{{\partial }^{2} {u}_{f}}{\partial {y}^{2}} + \frac{{\partial }^{2} {u}_{f}}{\partial {z}^{2}} ) \\ \frac{\partial ( {ρ}_{f} {v}_{f} )}{\partial t} + {u}_{f} \frac{\partial ( {ρ}_{f} {v}_{f} )}{\partial x} + {v}_{f} \frac{\partial ( {ρ}_{f} {v}_{f} )}{\partial y} + {w}_{f} \frac{\partial ( {ρ}_{f} {v}_{f} )}{\partial z} = - \frac{\partial p}{\partial y} + μ ( \frac{{\partial }^{2} {v}_{f}}{\partial {x}^{2}} + \frac{{\partial }^{2} {v}_{f}}{\partial {y}^{2}} + \frac{{\partial }^{2} {v}_{f}}{\partial {z}^{2}} ) \\ \frac{\partial ( {ρ}_{f} {w}_{f} )}{\partial t} + {u}_{f} \frac{\partial ( {ρ}_{f} {w}_{f} )}{\partial x} + {v}_{f} \frac{\partial ( {ρ}_{f} {w}_{f} )}{\partial y} + {w}_{f} \frac{\partial ( {ρ}_{f} {w}_{f} )}{\partial z} = - \frac{\partial p}{\partial z} + μ ( \frac{{\partial }^{2} {w}_{f}}{\partial {x}^{2}} + \frac{{\partial }^{2} {w}_{f}}{\partial {y}^{2}} + \frac{{\partial }^{2} {w}_{f}}{\partial {z}^{2}} ) + g ( {ρ}_{f} - {ρ}_{f , 0} ) \qquad ( 17 )$$

where p is the fluid pressure, p_{f,0} is the density of the fluid without contrast agent and g is the gravitation constant.

Solute Transport

Finally, solute transport was governed by the scalar convection-diffusion equation:

$$\frac{\partial C}{\partial t} + \frac{\partial ( {u}_{f} C )}{\partial x} + \frac{\partial ( {v}_{f} C )}{\partial y} + \frac{\partial ( {w}_{f} C )}{\partial z} = D ( \frac{{\partial }^{2} C}{\partial {x}^{2}} + \frac{{\partial }^{2} C}{\partial {y}^{2}} + \frac{{\partial }^{2} C}{\partial {z}^{2}} ) , \qquad ( 18 )$$

where C is the concentration of X-ray contrast agent in the fluid and D is the diffusion coefficient. The fluid density was dependent on the concentration of contrast agent (in density units, [g cm⁻³]):

ρ_{f}=ρ_{f,0}+C (19)

Model Geometry and Mesh

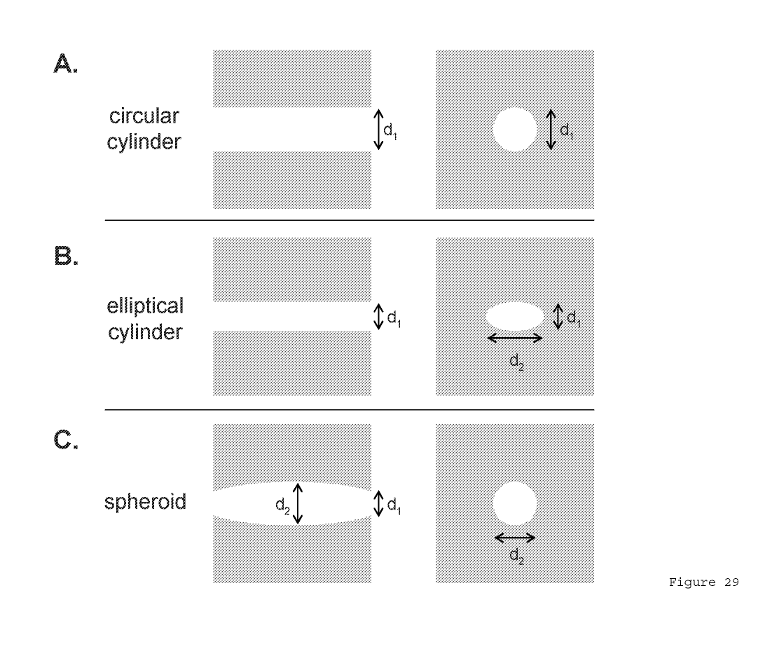

Consistent with the scaffold geometries obtained by micro-CT, cubic scaffolds with dimensions 4.5×4.5×4.5 mm³ containing single channels in the middle were modeled. Three different type channels were modeled (FIG. 29). The first type was a cylindrical channel with circular cross-section. The influence of channel diameter was examined by modeling four different diameters. The second channel type was a cylindrical channel with elliptical cross-section. Scaffolds with this channel type were compressed along the minor axis and the major axis of the elliptical cross-section. The third channel type consisted of an oblong spheroid. An overview of the channel dimensions is given in Table 3.

TABLE 3
Geometrical dimensions
of the scaffold pores and number of mesh elements

| Channel | | | # of solid | # of fluid |
|---|---|---|---|---|
| shape | d₁ | d₂ | elements | elements |
| Circular pore | 0.37 mm | — | 1003 | 3460 |
| | 0.94 mm | — | 461 | 3485 |
| | 1.38 mm | — | 425 | 2448 |
| | 1.89 mm | — | 811 | 1771 |
| Elliptic | 1.75 mm | 0.54 mm | 645 | 4424 |
| pore | | | | |
| Spheroid | 1.89 mm | 0.73 mm | 1379 | 3724 |

To reduce computational time, model symmetry was used. One symmetry plane was in the middle of the scaffold and perpendicular to the channel axis, and one symmetry plane was in the middle of the scaffold and parallel to the channel axis. Hence, only a quarter of the scaffold was modeled. To take into account the fluid surrounding the scaffold, a box was modeled with an interface to the channel domain. The model dimensions of this fluid reservoir were 4.5×2.25×2.25 mm³ (height×width×depth).

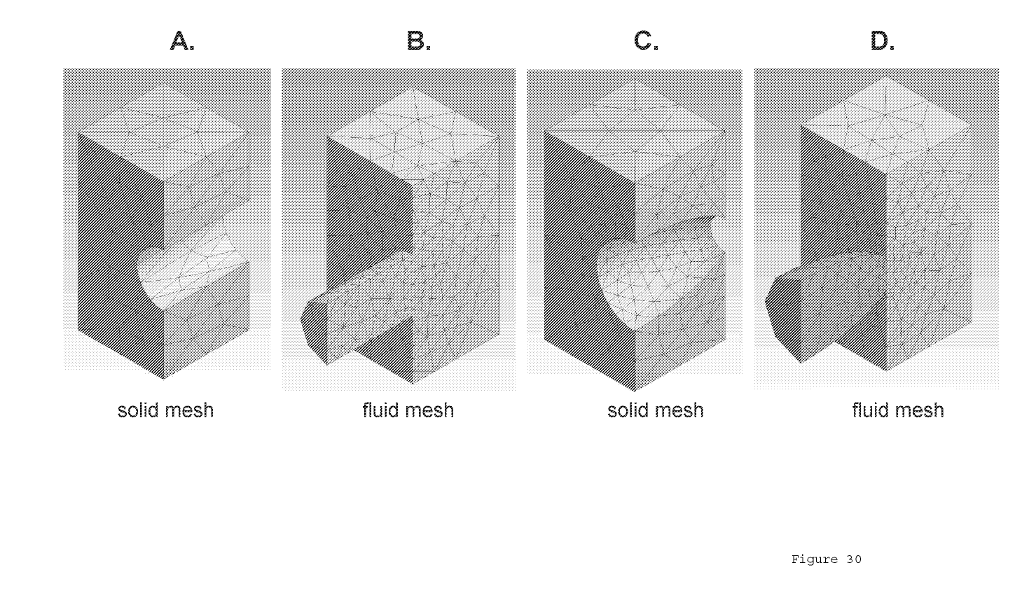

Both solid and fluid phases were meshed with tetrahedral elements. In the solid mesh, a refinement towards the fluid-solid interface was included to obtain more accuracy in displacements near the channel wall. The two fluid domains (channel and reservoir) were separately meshed to allow for different initial conditions in these domains. In the reservoir, the elements were increased in size away from the channel into the fluid reservoir. Typical solid and fluid meshes are shown in FIG. 30. Information about geometrical dimensions of the scaffolds and mesh statistics can be found in Table 3.

Boundary Conditions

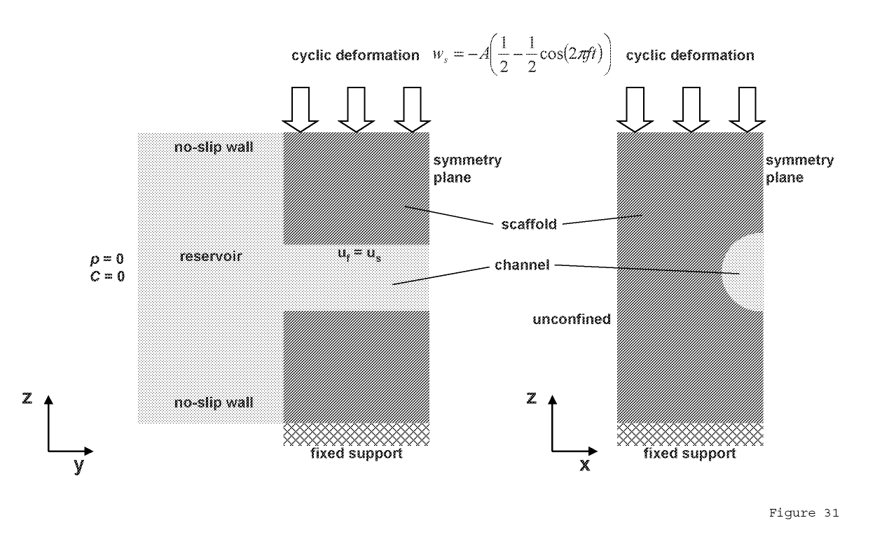

An overview of boundary conditions can be found in FIG. 31. The solid phase (scaffold) was subjected to the following boundary conditions:
The displacement of the bottom face of the scaffold was set to zero in all directions (i.e. uₛ=vₛ=wₛ=0)
The top face of the scaffold was cyclically compressed such that the vertical displacement wₛ followed a cosine wave:

$${w}_{s} = - A ( \frac{1}{2} - \frac{1}{2} cos ( 2 π f t ) ) , \qquad ( 20 )$$

where A is the compression amplitude and f is the compression frequency.
At the two symmetry planes, the displacement perpendicular to the symmetry plane was set to zero
The displacement at the interface between the scaffold and the fluid channel was used as a boundary condition in the fluid model. One-way coupling was used, i.e. it was assumed that the fluid pressure and shear acting at the scaffold-channel interface during compression was negligible compared to the stresses in the scaffold material as a result of the deformation.
The side faces of the scaffold were unconfined. Any fluid motion in the surrounding fluid reservoir due to deformation of the side faces was neglected.

The fluid phase (pores and fluid reservoir) was subjected to the following boundary conditions:
At the (deforming) walls of the channel, no-slip boundary conditions were implemented, meaning that the fluid velocity at the wall equals the time-derivative of the scaffold material displacement at the wall, i.e. solid wall velocity.
It was assumed that the scaffold material did not absorb any of the contrast agent. Therefore, the solute flux through the solid channel wall was set to zero (n·∇C=0, with n the vector normal to the channel wall).
The side of the fluid reservoir farthest from the channel opening was set to a zero gauge pressure boundary condition, i.e. p=0. The solute concentration was also set to zero at this boundary (C=0).
At the upper and lower faces of the fluid reservoir, no-slip wall boundary conditions were implemented.
At the two symmetry planes, the fluid velocity and the solute gradient normal to the symmetry plane were assumed to be zero (n·u_{f}=0 and n·∇C=0, where n is the vector normal to the symmetry plane).

Initial Conditions and Model Parameters

At t=0, the scaffold was assumed to be at rest and the fluid pressure and velocity were set to zero in the entire fluid domain. The concentration of solute was set to zero in the fluid reservoir and to C₀ inside the channel. Model parameters are summarized in Table 4.

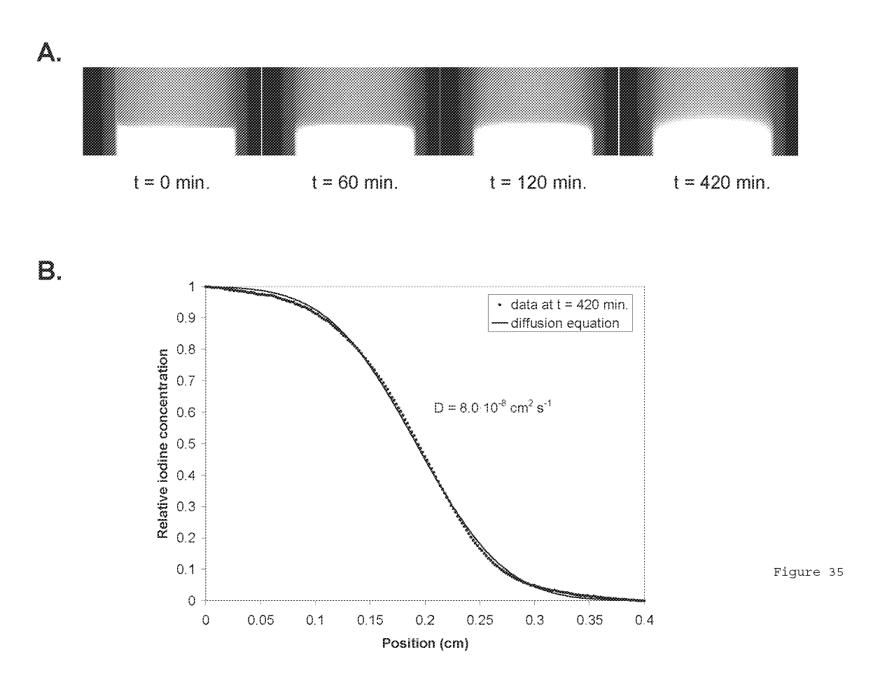

The diffusion coefficient of sodium iodine in glycerol was measured by carefully pouring a layer of the NaI glycerin solution in a vial containing glycerin only. This created a two-layer system, with NaI glycerin at the bottom. The upward diffusion of NaI from the bottom layer into the top layer was imaged using projection X-ray over a time period of 7 hours (FIG. 35). To quantify the diffusion coefficient, a line profile of the density values along the longitudinal axis of the vial was measured after 7 hours. The following analytical solution of the 1D-diffusion equation was then be fitted to this line-profile:

$$\frac{C}{{C}_{0}} = \frac{1}{2} - \frac{1}{2} erf ( \frac{x - {x}_{0}}{2 \sqrt{Dt}} ) , \qquad ( 21 )$$

where C/C₀ is the NaI concentration relative to the initial concentration in the bottom layer, and x is the absolute position with x₀ the position of the two-layer interface. Furthermore, D is the diffusion coefficient, t is the time and erf is the error-function. D was calculated (8.0.10⁻⁸ cm² s⁻¹) using the Levenberg-Marquardt nonlinear least-squares curve fitting algorithm with both x₀ and D as adjustable parameters (FIG. 35).

TABLE 4
Model parameter values

| Parameter | Value | Description | Reference |
|---|---|---|---|
| ρₛ | 1.12 g ml⁻¹ | Density | Kim et al., Nat. |
| | | scaffold | Biotechnol., 17: 979-83 |
| | | material | (1999) |
| E | 9.31 MPa | Young's | Kim et al., Nat. |
| | | modulus | Biotechnol., 17: 979-83 |
| | | | (1999) |
| υ | 0.5 | Poisson's ratio | Kim et al., Nat. |
| | | | Biotechnol., 17: 979-83 |
| | | | (1999) |
| ρ_{f,0} | 1.26 g ml⁻¹ | Fluid density | Ignatius et al., |
| | | | Biomaterials, 26: 311-8 |
| | | | (2005) |
| μ | 1.0 Pa s | Fluid viscosity | Ignatius et al., |
| | | | Biomaterials, 26: 311-8 |
| | | | (2005) |
| D | 8.0 · 10⁻⁸ cm² s⁻¹ | Diffusion | Described herein |
| | | coefficient | |
| C₀ | 31 mg ml⁻¹ | Initial | Liang et al., J. Biomech., |
| | | concentration | 41: 2906-11 (2008) |
| | | channel | |
| f | 1.0 Hz | Frequency of | Liang et al., J. Biomech., |
| | | compression | 41: 2906-11 (2008) |

Numerical Implementation

The commercial software ANSYS Workbench 11.0 (ANSYS Inc., Canonsburg, Pa.) was used to obtain numerical solutions. In this software, the dynamic solid deformation problem was solved using the Finite Element Method (FEM), whereas the Navier-Stokes and scalar transport equations were solved using the Finite Volume Method (FVM). At each time step, the solid deformation problem was solved first, and the mesh displacement at the fluid-solid interface was then transferred to the fluid solver as a boundary condition in the fluid flow problem. A fixed time step of t=0.025 s was found to be sufficiently small to obtain stable numerical results. Transient simulations were conducted until 100 s (simulation time) during cyclic compression or until 300 s without cyclic compression. The simulations were carried out on a 2.4 GHz AMD Opteron server with 16 GB memory running SUSE Linux 9. Computation time per run was approximately 5 hours.

Results

Passive Removal

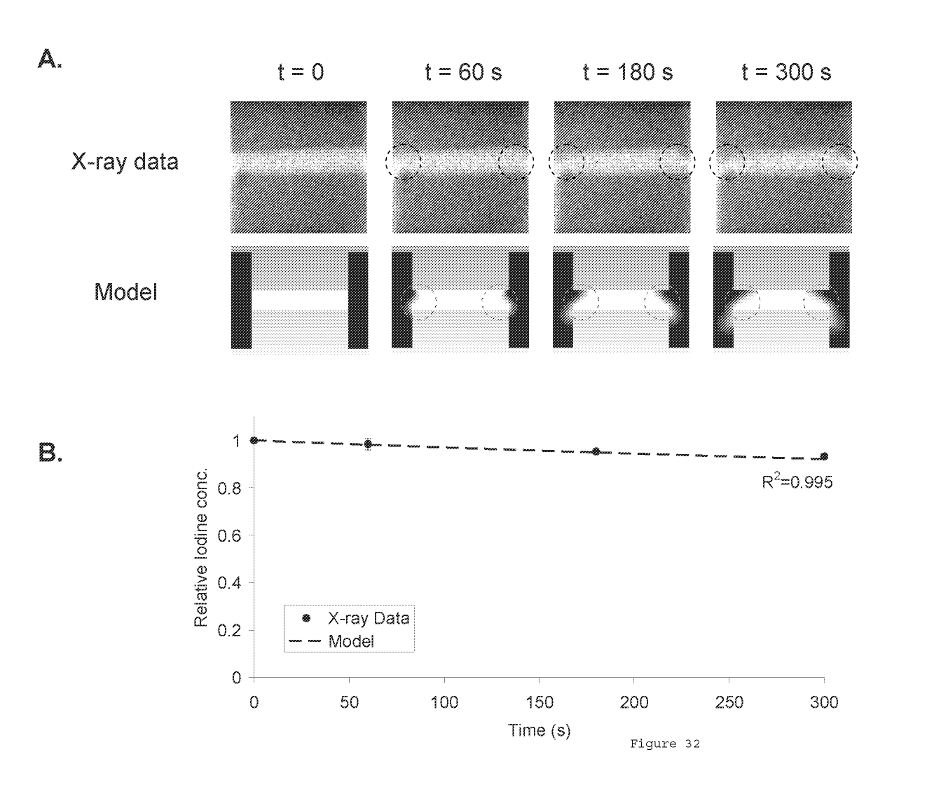

Without application of compression, natural convection as a result of gravitation, more so than diffusion, slowly removes some of the denser contrast agent at the openings of the channel into the surrounding fluid reservoir. This effect is captured in the model by including the gravitational term in the momentum equation (Eq. 17). FIG. 32A compares X-ray images with model simulations of the change in contrast agent distribution due to this ‘passive’ removal. Quantification of the image data shows that model simulations and experimental data agree well (FIG. 32B).

Compression-Induced Removal

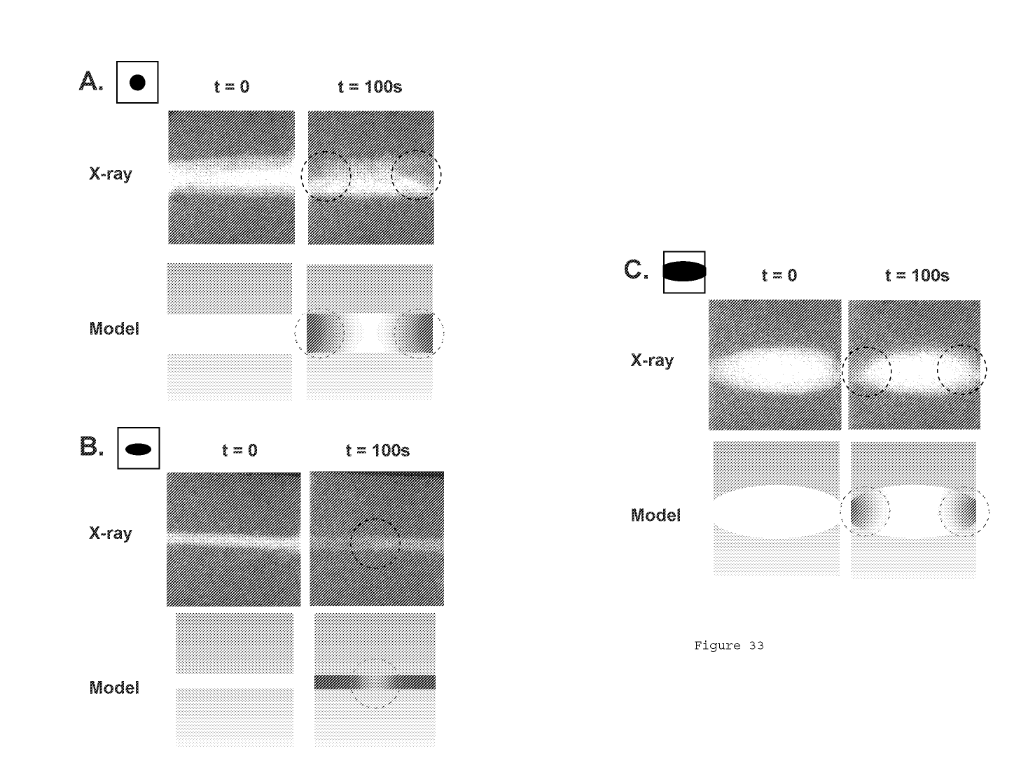

Upon application of 8.6% cyclic compression to the scaffolds at 1.0 Hz, the contrast agent inside the scaffold channel is dispersed into the surrounding fluid reservoir. FIG. 33 shows X-ray and computed images of the spatial distributions of the contrast agent inside different channels at t=0 (i.e., right after injection of the contrast agent into the channel) and after 100 cycles of compression at 1.0 Hz. The distributions obtained by the model are compared to the X-ray data.

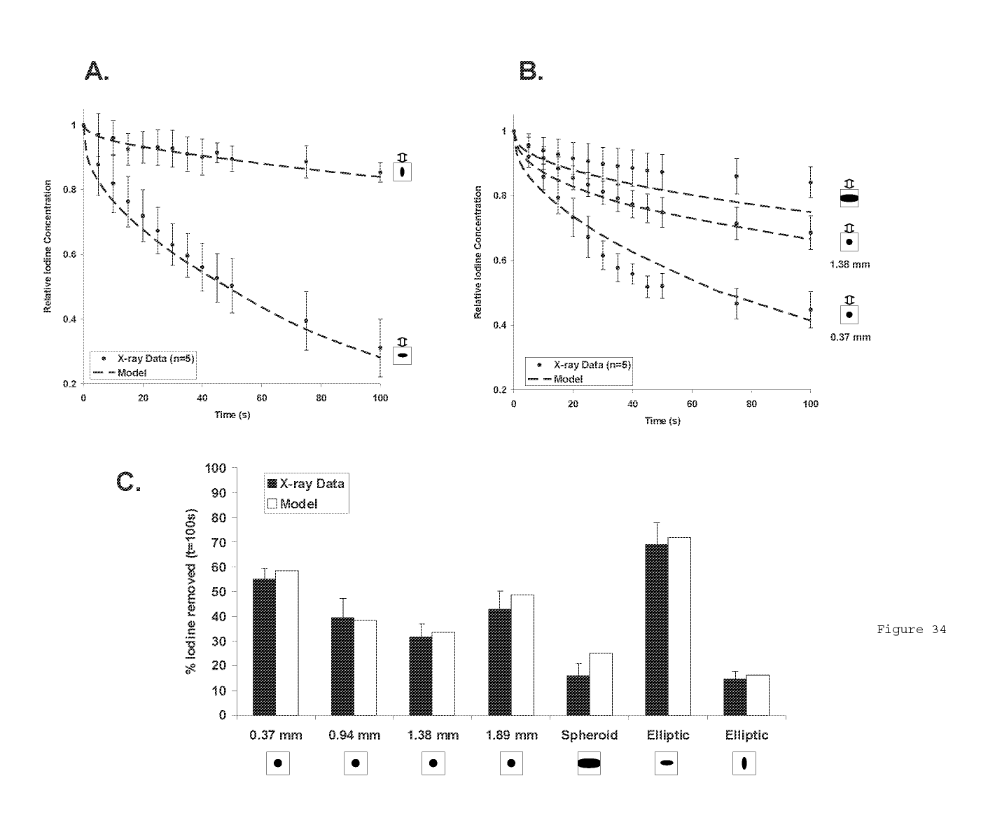

The model simulations were quantitatively compared to the experimental data by computing the average iodine concentration inside the pores. FIG. 34A shows the very good agreement of the model simulations with the data for the pore with elliptic cross-section, as compressed along its minor and major axis. Note that changing the direction of cyclic strain yields very different transport rates for this scaffold. FIG. 34B compares the model simulations to the experimental data for the spheroid pore, the circular pores with d=0.37 mm and d=1.38 mm. The model slightly overestimates the compression-induced removal for the scaffold with the spheroidal pore. FIG. 34C illustrates the excellent agreement of model and data for the percentage of iodine removed after 100 s for the different channels. The different pore geometries show very different rates of solute transport under cyclic compression, indicating the absolute importance of incorporating the pore geometry in the computational model.

The validated model was used to explore the effect of altering the diffusion coefficient D, the fluid viscosity μ and density ρ_{f}, and the maximum solute concentration C₀ (Table 5). These simulations were carried out for the scaffold with the 1.0 mm circular pore with a compression of 8.6% at 1.0 Hz. For the diffusion coefficient, values that are typical for physiologically relevant solutes such as albumin, glucose, and oxygen were explored. As expected, higher diffusion coefficients resulted in lower solute concentrations at t=100 s, although this effect was found to be minimal. For the viscosity, we explored a range of values, among which those for plasma and blood. Interestingly, the solute removal did not appear to be sensitive to the viscosity for the range of values simulated. The model also appeared to be insensitive to neglecting the density difference caused by the initial solute concentration C₀ as a result of the heavier X-ray tracer, and to reducing the fluid density to a value representative for water (instead of glycerol). Taken together, these simulations increase our confidence that the results obtained under the experimental conditions (using iodine in glycerol) are also relevant for physiological conditions (e.g., involving albumin, glucose or oxygen in water, plasma or blood).

TABLE 5
Model predicted relative concentrations for different diffusion
coefficients, fluid viscosities, and gravitational effects

| | | | C/C₀ |
|---|---|---|---|
| D (cm² s⁻¹) | C/C₀ (t = 100 s) | μ (mPa s) | (t = 100 s) |
| 8.0 · 10⁻⁸ | 0.657 | 0.89 (water) | 0.657 |
| 9.4 · 10⁻⁷ (albumin) | 0.655 | 1.4 (plasma) | 0.670 |
| 6.9 · 10⁻⁶ (glucose) | 0.641 | 3.1 (blood) | 0.678 |
| 2.4 · 10⁻⁵ (oxygen) | 0.599 | 100 | 0.644 |
| | | 1000 (glycerol) | 0.640 |

| C₀ (mg ml⁻¹) | ρ_{f} (g ml⁻¹) | C/C₀ (t = 100 s) |
|---|---|---|
| 31 | 1.26 | 0.640 |
| 0 | 1.26 | 0.652 |
| 0 | 1.00 | 0.652 |

These data demonstrate that a thoroughly validated computational model of fluid and mass transport in cyclically deforming scaffold pores can be developed. In the present study, a fluid-structure interaction model of solute transport in deformable scaffolds with pores of different shapes and dimensions was developed and validated. The model was in agreement with experimentally obtained X-ray imaging data of a contrast agent inside the pores of cyclically deformed biodegradable scaffolds. The significant impact of pore shape and orientation of the pore cross-section with respect to the direction of strain demonstrated that pore geometry is an important factor in a computational model of solute transport in the pores of deformable scaffolds. Considering the increasing use of rapid prototyping technologies to manufacture scaffolds with pre-designed pore architectures (see, e.g., Lu and Mikos, MRS Bull 21:28-32 (1996)), a geometry-based computational model of solute transport can accelerate iterative design processes of scaffolds by rapidly evaluating a number of designs in silica, thereby limiting the costly and time consuming experimental evaluation to only the most promising candidate pore architectures.

Previous models of cyclically compressed porous media that use the biphasic mixture theory generally incorporate the pore geometry into Darcy's permeability constant, which is a statistical average (Mauck et al., J. Biomech. Eng., 125:602-14 (2003); Gardiner et al., Comput. Methods Biomech. Biomed. Engin., 10:265-78 (2007); and Sengers et al., J. Biomech. Eng., 126:82-91 (2004)). Darcy's law can be used to relate the average fluid velocity u_{f} through the scaffold to the pressure difference across the scaffold ΔP via the permeability constant K and the fluid viscosity μ:

$${u}_{f} = - \frac{K}{μ} ∇ P \qquad ( 22 )$$

The permeability K is a function of the scaffold pore architecture and porosity, and has proven a useful predictor for biological outcomes such as tissue ingrowth. For example, it was shown in cancellous bone grafts implanted in rabbits, that a threshold permeability exists, below which revascularisation and the formation of osteoblasts and fibrous tissues could not be attained (Hui et al., J. Biomech., 29:123-32 (1996)). High scaffold permeability as provided by well interconnected pores is suggested to be essential to provide the space for vascular tissue ingrowth followed by new tissue formation (Li et al., Biomaterials, 28:2810-20, (2007) and Mastrogiacomo et al., Biomaterials, 17:3230-7 (2006)). The use of a lumped parameter for permeability is computationally efficient, especially when dealing with scaffold pore geometries that are difficult to model, which is often the case in hydrogels or scaffolds fabricated using a particulate-leaching or gas-foaming method. However, with the emerging availability of solid freeform fabrication methods it becomes possible to program the scaffold pore geometry with computer-aided design (CAD) software (see, e.g., Hollister, Nat. Mater., 4:518-24 (2005)), such that the scaffold mass transport properties are reproducible and more accurately predictable at the pore level before actual fabrication of the scaffold. These accurate predictions require a thoroughly validated, three-dimensional numerical model of mass transport in the scaffold pores, incorporating the precise pore geometry from the initial CAD data. Such a model will allow for the rapid exploration of transport properties of a wide range of pore architectures before actual fabrication and experimental testing.

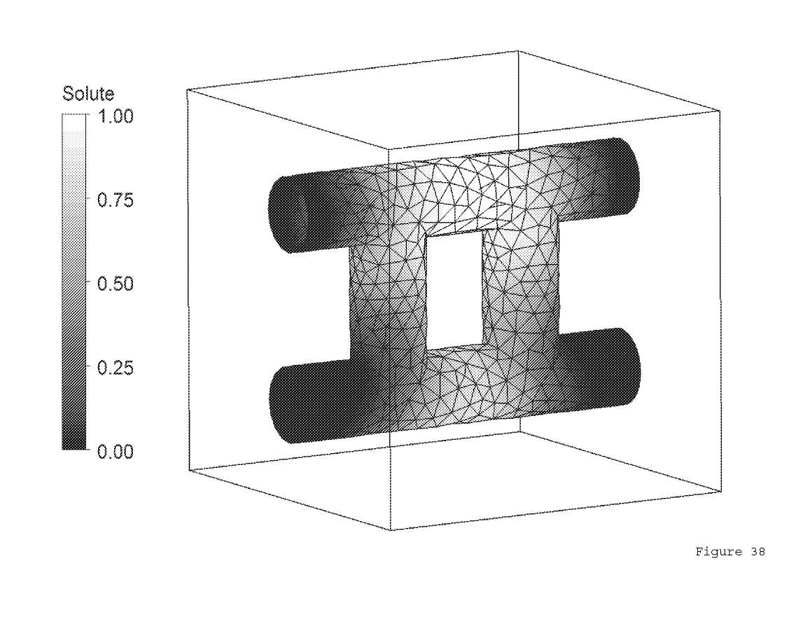

The current model contains several simplifications, which were made to reduce computation time and/or because additional parameters were lacking First, the scaffold deformation model pushes the limits of the small strain theory at the ˜10% strain used here. In addition, the scaffold material characteristics were assumed to be linear and isotropic, while a nonlinear stress-strain curve is often observed in polymers. If a more elaborate description of the mechanical behavior of the scaffold is desired, these limitations can be addressed by including large deflection theory and/or a more complex material model. Considering the coupling of solid and fluid, the model assumed one-way coupling, i.e., the pressure of the fluid acting on the scaffold wall was neglected. Two-way coupling, i.e., including the force of the fluid acting on the scaffold material, may need to be included when scaffolds of higher porosity with thinner membranes of scaffold material are modeled. In this case, the fluid pressure and shear stresses may have a significant impact on the scaffold matrix deformation. In this example, scaffolds with single straight pores with various shapes were used, whereas more realistic scaffolds would have pore labyrinths comprised of interconnected channels in three dimensions. However, the modeling approach possesses the capability to describe more complex 3D pore networks, as it is based on the exact geometry of the scaffold design. FIG. 38 demonstrates the feasibility to model transport in dynamically deforming interconnected channels. Describing more complex pore systems with the present model can be limited by computational hardware.

With respect to the fluid model, laminar flow was assumed, which seems a reasonable assumption considering the small pore dimensions. The Reynolds's number (Re) is given by:

$$Re = \frac{{ρ}_{f} {u}_{f} d}{μ} \qquad ( 23 )$$

where d is the channel diameter. Assuming water as the fluid (p_{f}=1.0·10³ kg m⁻³ and μ=1.0·10⁻³ Pa s), a fluid velocity of u_{f}=5 mm s⁻¹, and a channel diameter of 1.0 mm, the value for Re=5. This is well below the Reynolds number at which a flow typically transitions to turbulence (Re=2000). Still, turbulence may occur at sharp transitions in the geometry or at higher compression frequencies and amplitudes. Turbulence could increase solute mixing and dispersion, and may be included in the model by e.g., the empirical k-ε model (Versteeg and Malalasekera, An introduction to computational fluid dynamics: the finite volume method, Harlow: Pearson Prentice Hall (2007)). At the scaffold-fluid interface, no-slip wall boundary conditions were assumed. Roughness of the scaffold surface was not taken into account, which may alter the fluid flow in regions near the scaffold wall. The fluid flow at the wall may have an impact on the distribution of solutes near the wall, and hence the nutrient supply of cells attached to the scaffold surface. Additionally, an improved fluid mesh in the model near the scaffold wall may yield a more accurate prediction of fluid shear stress, which in addition to nutrient supply could have an important effect on the proliferation, distribution and differentiation of cells. Despite these limitations, the current model yielded a good quantitative prediction of the average solute transport rate in scaffolds with different pore geometries, and could be further improved if more accurate quantitative information on e.g., scaffold mechanical behavior, turbulence and wall shear stress is desired.

These data demonstrate that cyclic compression can increase solute convection by cyclic fluid motion and subsequent spreading of solutes dissolved in the fluid. The efficiency of the cyclic pumping can be increased by considering the strain direction and manufacturing pores with highly deformable cross-sectional geometries. Thus, careful design and fabrication of deformable porous tissue scaffolds may be a strategy through which solutes can be transported beyond the diffusion limit after implantation of constructs with clinically relevant thicknesses. The proposed computational model will aid such scaffold design.

### Example 6

### Design and Construction of Synthetic Porous Scaffolds

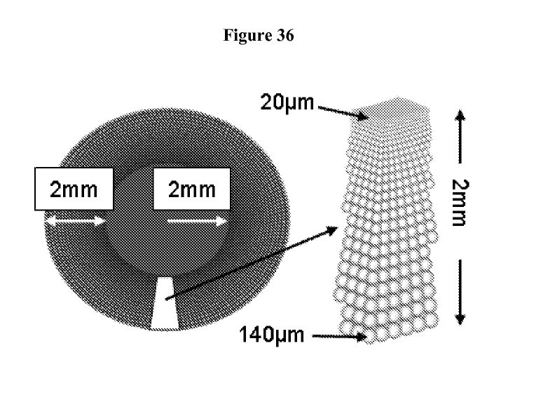

The following is conducted to design and construct a synthetic arterial conduit suitable for ultimate replacement of a vessel segment by ingrowing tissue. The design of the arterial wall is such that it mimics several of the solute transport features of the natural arterial wall. The natural arterial wall is made up of concentric layers of tissue that have different mechanical properties and it has vasa vasorum, most of which enter and leave via the adventitia. The media has a plexus of capillaries which bring nutrients and wash out metabolic products as well as remove solutes that migrate across the arterial wall due to the pressure gradient across the wall. These features can be fairly well replicated by concentric layers of microspheres made up of scaffold material (see FIG. 36), with the spaces between them serving as channels for solute transport and cellular and/or vascular invasion. The arterial wall can be sufficiently flexible so that the arterial pulsation will provide the cyclic compression needed to maintain solute transport within the pores. The wall is generated with a printer that creates a hollow “thick walled” cylinder with the wall made up of, in effect, sintered microspheres. The spheres at the lumen/wall interface are about 20 μm diameter so that the spaces between them will not let red blood cells through. The microspheres are of increasing diameter as the layers progress away from the lumen such that the outer layer will be made of 140 μm diameter microspheres which will have spaces between them that will allow microvessels to grow in. The outer microspheres are made stiffer than the inner microspheres so that compression will be preferentially in the inner layers, thereby functioning somewhat as a pump that removes solutes diffusing out of the main lumen into the “intima.”

### Example 7

### Design and Construction of Injectable Synthetic Porous Scaffolds

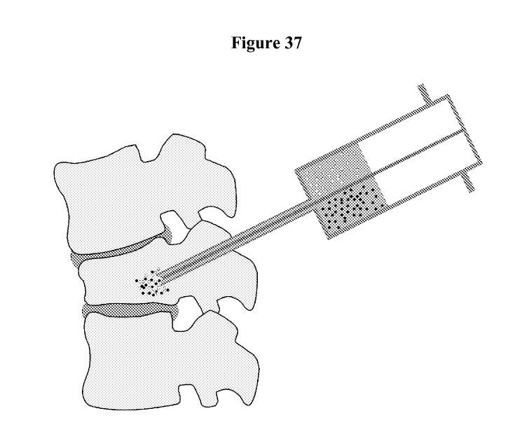

The following is conducted to design and construct an injectable porous scaffold suitable for ultimate replacement by ingrowing tissue components. The concept of an injectable scaffold is schematically demonstrated in FIG. 37. The scaffolds are designed and generated to be injected in liquid form into their intended position and then to solidify to produce scaffolds that have interconnected pores suitable for maintaining viable cells and thus be ultimately replaced by ingrowing tissue. The resulting scaffold can have the ability to maintain convective flow within the pores caused by slight repetitive distortion of the somewhat pliable scaffold material. Three types of microsphere (<300 μm diameter) suspensions are injected:

(1) Solid spheres that bond to each other where they come in contact and thereby form the scaffold. The fluid-filled spaces between them form the pores. These spheres have a random packing for porosity to exceed the approximately 40% of “crystalline” porosity. Preliminary studies show that 50% porosity can be readily achieved.

(2) Porous microspheres instead of solid spheres. Porous microspheres permit increasing the porosity even if the packing of the spheres is “crystalline.”

(3) Degradable microspheres, which when packed flatten somewhat and bond there as they touch, which allows the material in the pores between the spheres to polymerize and become the scaffold.

Microspheres are printed in a crystalline or more contrast-containing fluid in the pores by our CT imaging method. Microspheres are injected into a pliable plastic bag, then the contents of that bag are exposed to vacuum so that the spheres are kept in place by friction due to outside pressure. This bag is then encapsulated in a cast (slightly elastic material) which maintains the external shape and volume of the packed microspheres so that the pores can now be filled with contrast-containing fluid. This specimen is imaged and the pore space visualized both statically and during a cyclic compression sequence. This interim approach temporarily avoids (delays) the immediate need to fully develop the technique of injecting the two components of the injectable scaffold and the need to develop the ‘click’ chemistry aspect of this approach. See, e.g., Evans et al., Chem. Commun., (17):2305-2307 (2009) and Zhang et al., Carbohydrate Polymers 77:583-589 (2009).

For the complete scaffold, the 3D images are subjected to finite element analysis so as to provide an estimate of mechanical properties of the scaffold. The algorithm which finds the path of least resistance to flow is used so that regions of inadequate convective transport can be identified. This information can then be compared to CT-based measurements of contrast transport within the porous structure.

A numerical model is developed which describes solute transport inside the pores of the scaffold wall. The model takes into account the deformation of the scaffold wall and hence of the pores, as a result of the arterial pressure pulse. This deformation influences the fluid and solute transport in the wall.

The deformation of the scaffold is modeled using a dynamic small-strain problem with a linear Hooke's law as constitutive equation, assuming linear, isotropic elastic behavior. Fluid pressure and velocity of the fluid phase in the pores are described by the Navier-Stokes equations, assuming an incompressible Newtonian fluid and laminar flow. Solute transport is modeled by the scalar convection-diffusion equation, where the convection is calculated from the Navier-Stokes equations. However, because the Reynolds numbers will be so low that likely only the Stokes equations will be relevant.

The topography of scaffold/pore system is obtained from the stereolithography files used to print the experimental scaffold, or from meshing the micro-CT images of the scaffolds (material) and their complement (the fluid) in the image processing software Mimics (Materialize, Ann Arbor, Mich.). To reduce computation time, a section (approximately 2 mm thick) of the artificial vessel is modeled. 3D 10-node tetrahedral elements with quadratic displacement behavior are used to mesh the scaffold geometry. The fluid domain is meshed using 3D tetrahedral elements with linear shape functions and integration points at the center of each surface. A convergence study is carried out to investigate the effects of mesh element size and time steps on the solution. Boundary conditions are applied such that the model accounts for the interaction between the fluid and the scaffold material. The fluid velocity at the wall of the scaffold is set equal to the velocity of the scaffold material at their interface. To mimic the arterial pressure wave, a pressure boundary condition in the form of a cosine wave is applied to the material surface on the inside of the arterial vessel.

The software Ansys Workbench is used to obtain numerical solutions. The dynamic solid deformation problem is solved using the Finite Element Method (FEM), and the Navier-Stokes and scalar transport equations will be solved using the Finite Volume Method (FVM). At each time step, the solid deformation problem is solved first, and the calculated displacement at the fluid-solid interface is then transferred to the fluid solver as a boundary condition in the fluid flow problem. The fluid velocity, pressure and solute concentration fields are solved using the CFD solver. The numerical solution procedure is carried out on a Sun server with 8 quad-core processors (2.66 GHz) and 256 GB internal memory, running SUSE Linux Enterprise Server 10. File I/O will be governed by a 10,000 rpm Raid 0 array with a disk space of 1.0 TB.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for supporting tissue growth within a mammal, wherein said method comprises implanting a tissue scaffold into a location in said mammal, wherein said location provides a compressive or expansive force to said tissue scaffold, wherein said force is generated from a natural body movement or body function.

2. The method of claim 1, wherein said mammal is a human.

3. The method of claim 1, wherein said tissue scaffold comprises a population of cells.

4. The method of claim 3, wherein said cells are selected from the group consisting of stem cells, preadipocytes, glia, fibroblasts, myocytes, and osteocytes.

5. The method of claim 1, wherein said tissue scaffold comprises a porous geometry for solute transport.

6. The method of claim 1, wherein said location is selected from the group consisting of the heart, intestines, vasculature, knee, hip, or jaw.

7. The method of claim 1, wherein said force is applied cyclically.

8. The method of claim 7, wherein said frequency of said force is equal to or greater than about 1.0 Hz.

9. The method of claim 8, wherein said force enhances solute transport within said tissue scaffold.

10. The method of claim 1, wherein said body function comprises beating of said mammal's heart, pulsation of said mammal's arteries, or peristaltic motion of said mammal's intestines.

11. A method for supporting tissue growth within a mammal, wherein said method comprises implanting a tissue scaffold into a location in said mammal, wherein said location provides a compressive or expansive force to said tissue scaffold, wherein said force is generated from a natural body movement or body function, and wherein said tissue scaffold comprises concentric layers.

12. The method of claim 11, wherein said mammal is a human.

13. The method of claim 11, wherein said tissue scaffold comprises a population of cells.

14. The method of claim 11, wherein said tissue scaffold comprises microspheres.

15. The method of claim 14, wherein said microspheres are selected from the group consisting of solid microspheres, porous microspheres, and degradable microspheres.

16. The method of claim 11, wherein said tissue scaffold comprises a porous geometry for solute transport.

17. The method of claim 11, wherein said body function comprises beating of said mammal's heart or pulsation of said mammal's arteries.

18. A method for supporting tissue growth within a mammal, wherein said method comprises injecting an injectable tissue scaffold material into a location in said mammal, wherein said location is substantially free from a compressive or expansive force, wherein said injectable tissue scaffold material forms a porous geometry for solute transport.

19. The method of claim 18, wherein said injectable tissue scaffold comprises microspheres.

20. The method of claim 18, wherein said location is within a vertebral body.
